(19) United States
(12) Patent Application Publication
(10) Pub. No.: US 2023/0329622 A1
(43) Pub. Date: Oct. 19, 2023

(54) SIMULATION PACEABLE HEART

(71) Applicants: Northwestern University, Evanston, IL (US); Ann & Robert H. Lurie Children's Hospital of Chicago, Chicago, IL (US)

(72) Inventors: Ahmad Sami Chaouki, Chicago, IL (US); Mary Eileen McBride, Chicago, IL (US); David Patrick O’Neill, Wilmette, IL (US); Ryan Fong, Evanston, IL (US); Isabella M. Rischall, Brooklyn, NY (US); Di Wu, Evanston, IL (US)

(21) Appl. No.: 18/043,173

(22) PCT Filed: Aug. 26, 2021
(86) PCT No.: PCT/US2021/047660

§ 371 Date: Feb. 27, 2023

Related U.S. Application Data
(60) Provisional application No. 63/071,405, filed on Aug. 28, 2020.

(51) Int. Cl. A61B 5/352; A61N 1/39; A61N 1/37; A61N 1/368

(52) U.S. Cl. CPC A61B 5/352; A61N 1/368; A61N 1/3706; A61N 1/39622; A61N 1/3704

(57) ABSTRACT

A cardiac conduction simulation system for pacemakers includes a memory configured to store one or more inputs from one or more users. The one or more inputs include a selection of an arrhythmia scenario for a pacemaker. The system also includes a processor operatively coupled to the memory and configured to generate an electrogram signal for the pacemaker based at least in part on the selected scenario. The processor is also configured to process one or more output pacing signals received from the pacemaker. The processor is further configured to generate an electrocardiogram (ECG) signal in accordance with the processed one or more output pacing signals from the pacemaker.

### CROSS REFERENCE TO RELATED APPLICATION

The present application claims the priority benefit of U.S. Provisional Pat. App. No. 63/071,405 filed on Aug. 28, 2020, the entire disclosure of which is incorporated by reference herein.

### BACKGROUND

A pacemaker refers to an electronic device that helps a patient’s heart beat more regularly through electric stimulation. Teaching pacemaker management to medical professionals can be challenging. Currently, knowledge comes from textbooks, seminars, and “on-the-fly” education while managing patients who may be critically ill or dependent on their devices. This leads to a lack of practice in a safe environment as well as subjecting patients to potential harm while learning how to manage their device. Because of this, few medical professionals can become proficient at management and this may put patients at risk if they require potentially lifesaving measures in pacing.

### SUMMARY

An illustrative cardiac conduction simulation system for pacemakers includes a memory configured to store one or more inputs from one or more users. The one or more inputs include a selection of an arrhythmia scenario for a pacemaker. The system also includes a processor operatively coupled to the memory and configured to generate an electrogram signal for the pacemaker based at least in part on the selected scenario. The processor is also configured to process one or more output pacing signals received from the pacemaker in response to the electrogram signal. The processor is further configured to generate an electrocardiogram (ECG) signal in accordance with the processed one or more output pacing signals from the pacemaker.

In some embodiments, the selected scenario comprises a heart condition that is affected by the pacemaker. In other embodiments, the one or more inputs include a parameter setting for the scenario, and the parameter setting includes an atrial rate and a ventricular rate. The parameter setting can also include atrial or ventricular association or dissociation, and in a PR interval in the case of ventricular association. In other embodiments, the parameter setting includes a P wave amplitude or an R wave amplitude. The parameter setting can also include one or more capture thresholds.

In some embodiments, the system includes a digital to analog converter (DAC) controlled by the processor, where the DAC is configured to generate the electrogram signal. In other embodiments, the processor is configured to temporally align the electrogram signal and the ECG signal. The system may also include an analog to digital converter (ADC) in communication with the processor, where the ADC is configured to measure of a voltage of the one or more output pacing signals of the pacemaker. In some implementations, an initial heart state is stored in the memory, and the processor transitions a simulation from the initial heart state to a subsequent heart state based at least in part on the one or more output pacing signals of the pacemaker that are received in response to the electrogram signal. In such an implementation, the ECG signal corresponds to the subsequent heart state.

The processor can also be configured to add electrical noise to the electrogram, mimicking real-life interference encountered by a pacemaker. The processor can further be configured to add one or more of digital noise, drift, and randomization to the ECG signal. The system can further include a display operatively coupled to the processor, where the display is configured to display the ECG signal, display the ECG effective heart rate, and display performance feedback data to the one or more users during or after completion of a simulation. Some embodiments include a pacemaker circuit that is configured to interface with the pacemaker, where the pacemaker circuit includes a load resistor configured to simulate resistance in a human heart. A sensing circuit that receives an output from the pacemaker circuit may also be included in the system. The sensing circuit includes an inverted operational amplifier to divide and invert the output from the pacemaker circuit such that a maximum voltage of the output is controlled. The sensing circuit can also include a non-inverting operational amplifier that is configured to rectify and amplify the output such that the output is maintained at a specific voltage.

An illustrative method of simulating cardiac conduction for pacemaker management includes storing, in a memory of a computing system, one or more inputs from one or more users. The one or more inputs include a selection of an arrhythmia scenario for a pacemaker. The method also includes generating, by a processor operatively coupled to the memory, an electrogram signal for the pacemaker based at least in part on the selected scenario. The method also includes processing, by the processor, one or more output pacing signals received from the pacemaker in response to the electrogram signal. The method further includes generating, by the processor, an electrocardiogram (ECG) signal in accordance with the processed one or more output pacing signals from the pacemaker.

In some embodiments, the method also includes storing, in the memory, a parameter setting for the scenario, where the parameter setting includes an atrial rate, a ventricular rate, atrial association or dissociation, ventricular association or dissociation, a P wave amplitude, an R wave amplitude, or one or more capture thresholds. The method can also include temporally aligning, by the processor, the electrogram signal and the ECG signal. The method can further include adding, by the processor, electrical noise to the electrogram signal.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts healthy theoretical intracardiac electrograms and their temporal relation to an ECG signal in accordance with an illustrative embodiment.

FIG. 2A is a schematic of a simulation model in accordance with an illustrative embodiment.

FIG. 2B is a schematic of a simulation model with an examination/proctored mode in accordance with an illustrative embodiment.

FIG. 3 is a block diagram depicting a design scheme for the simulation system in the form of a closed-loop system in accordance with an illustrative embodiment.

FIG. 4 depicts a simulated normal sinus rhythm ECG signal at 120 beats per minute (bpm) in accordance with an illustrative embodiment.

FIG. 5 depicts a realistic electrogram (top) and a simulated impulse electrogram (bottom) plotted in MATLAB to visualize differences in shape in accordance with an illustrative embodiment.

FIG. 6 depicts an ECG generation code workflow in accordance with an illustrative embodiment.

FIG. 7 depicts an ECG displayed in a user interface application during a simulation in accordance with an illustrative embodiment.

FIG. 8 depicts a user interface application screen shot of operational modes in accordance with an illustrative embodiment.

FIG. 9A depicts how a linear model was fitted to the data to determine the relationship between required DAC output and pacemaker sensitivity setting in accordance with an illustrative embodiment.

FIG. 9B is a table depicting data corresponding to the linear model of FIG. 9A in accordance with an illustrative embodiment.

FIG. 10A depicts how a linear model was fitted to the data to determine the relationship between actual and expected voltages in accordance with an illustrative embodiment.

FIG. 10B is a table depicting data corresponding to the linear model of FIG. 10A in accordance with an illustrative embodiment.

FIG. 11 depicts a pacemaker circuit in accordance with an illustrative embodiment.

FIG. 12 depicts a sensing circuit in accordance with an illustrative embodiment.

FIG. 13 depicts a measurement circuit of the system in accordance with an illustrative embodiment.

FIG. 14A is a table that depicts ECG component amplitudes, starting times, and durations for pediatric normal sinus rhythm in accordance with an illustrative embodiment.

FIG. 14B is a table that depicts atrial electrogram component amplitudes, starting times, and durations for pediatric normal sinus rhythm in accordance with an illustrative embodiment.

FIG. 14C is a table that depicts ventricular electrogram component amplitudes, starting times, and durations for pediatric normal sinus rhythm in accordance with an illustrative embodiment.

FIG. 14D is a table that depicts ECG component amplitudes, starting times, and durations for pediatric sinus bradycardia in accordance with an illustrative embodiment.

FIG. 14E is a table that depicts atrial electrogram component amplitudes, starting times, and durations for pediatric sinus bradycardia in accordance with an illustrative embodiment.

FIG. 14F is a table that depicts ECG component amplitudes, starting times, and durations for pediatric 1st degree heart block in accordance with an illustrative embodiment.

FIG. 14G is a table that depicts atrial electrogram component amplitudes, starting times, and durations for pediatric 1st degree heart block in accordance with an illustrative embodiment.

FIG. 14H is a table that depicts ventricular electrogram component amplitudes, starting times, and durations for pediatric 1st degree heart block in accordance with an illustrative embodiment.

FIG. 14I is a table that depicts ECG component amplitudes, starting times, and durations for pediatric Mobitz type 1, 2nd degree heart block in accordance with an illustrative embodiment.

FIG. 14J is a table that depicts atrial electrogram component amplitudes, starting times, and durations for pediatric Mobitz type 1, 2nd degree heart block in accordance with an illustrative embodiment.

FIG. 14K is a table that depicts ventricular EG component amplitudes, starting times, and durations for pediatric Mobitz type 1 2nd degree heart block in accordance with an illustrative embodiment.

FIG. 14L is a table that depicts ECG component amplitudes, starting times, and durations for pediatric Mobitz type 2, 2nd degree heart block in accordance with an illustrative embodiment.

FIG. 14M is a table that depicts atrial electrogram component amplitudes, starting times, and durations for pediatric Mobitz type 2, 2nd degree heart block in accordance with an illustrative embodiment.

FIG. 14N is a table that depicts ventricular electrogram component amplitudes, starting times, and durations for pediatric Mobitz type 2, 2nd degree heart block in accordance with an illustrative embodiment.

FIG. 14O is a table that depicts ECG component amplitudes, starting times, and durations for pediatric atrial flutter in accordance with an illustrative embodiment.

FIG. 14P is a table that depicts atrial electrogram component amplitudes, starting times, and durations for pediatric atrial flutter in accordance with an illustrative embodiment.

FIG. 14Q is a table that depicts ventricular electrogram component amplitudes, starting times, and durations for pediatric atrial flutter in accordance with an illustrative embodiment.

FIG. 15 is a flow diagram of a decision tree for atrial events in accordance with an illustrative embodiment.

FIG. 16 is a flow diagram of a decision tree for ventricular events in accordance with an illustrative embodiment.

FIG. 17 depicts ECG signals for developed rhythms in accordance with an illustrative embodiment.

FIG. 18 is a scenario selection page of the UI application in accordance with an illustrative embodiment.

FIG. 19 is a sinus bradycardia parameters page of the user interface in accordance with an illustrative embodiment.

FIG. 20 is an atrial EG parameters page of the user interface in accordance with an illustrative embodiment.

FIG. 21 is a ventricular EG parameters page of the user interface in accordance with an illustrative embodiment.

FIG. 22 depicts a graphing page in accordance with an illustrative embodiment.

FIG. 23 is a post simulation selection page of the user interface in accordance with an illustrative embodiment.

FIG. 24 is a block diagram of a computing system to implement the cardiac conduction simulation system in accordance with an illustrative embodiment.

### DETAILED DESCRIPTION

Traditional pacemaker training for medical personnel includes attendance of a seminar outlining general pacemaker use, followed by doctor-supervised, hands-on training sessions with live patients. Unfortunately, the number of training opportunities varies with doctor availability. Also, inexperienced use of pacemakers on a live patient risks harming the patient. Traditional pacemaker training techniques also limit opportunities for emergency response training because emergency scenarios occur infrequently. Therefore, traditional training methods fail to provide a comprehensive and safe training regime for incoming medical providers.

As cardiac surgery grows more complex, the risks of postoperative arrhythmias increase. Temporary pacing is required for potentially life-saving postoperative management, however managing arrhythmias is complex and difficult to teach. Often these arrhythmias occur in the most fragile of patients and there is substantial risk if pacemaker programming is incorrect. There are currently no commercially available high fidelity simulation devices that allow for learning temporary pacemaker management in a safe environment. Described herein is a system that aims to simulate a cardiac conduction system with outputs that mimic the human heart and the ability to receive signals from any temporary or permanent pacemaker.

The system described herein is a simulation device that trains medical providers for pacemaker use without patient involvement. Currently, medical practitioners have limited hands-on training because these opportunities vary with doctor and patient availability. Because inexperienced use of pacemakers may harm patients, medical trainees may be inadequately prepared for emergency scenario responses. The inventors have raised the need to provide a comprehensive and safe external pacemaker training regimen for medical providers. Specifically, described herein is a simulation-based training system that eliminates the need for patient involvement. No commercial solutions exist for this problem.

The proposed simulation system can act as an automated rhythm management simulator. The system is a simulation cardiac conduction system that allows for safe teaching of temporary and permanent pacemaker programming. It provides realistic rhythm disturbances and responds to pacemaker inputs. The educator can program any imaginable rhythm. The device will output true to life electrograms that can be received by a real temporary or permanent pacemaker. This allows the learner to identify the rhythms and characteristics of pacing leads and decide how to best program a pacemaker in a safe manner. In addition, the system is capable of producing issues that occur in clinical settings that can be disruptive to pacing such as electrical interference and other sensing issues. The system responds to inappropriate pacing in a realistic manner with the potential of inducing different arrhythmias.

In a manual mode, the educator (or proctor) can program all characteristics, including atrial and ventricular rates, AV association or dissociation, P and R wave amplitudes and capture thresholds. This can allow for any rhythm that can potentially be encountered. In automatic modes, the system can randomly generate clinical scenarios. These rhythms include bradycardia such as sinus bradycardia, junctional bradycardia, second and third degree heart block, etc. Tachycardia management can also be achieved by programming reentrant arrhythmias such as atrial flutter, atrioventricular reentrant and atrioventricular nodal reentrant tachycardia that can be pace terminated, etc. Junctional ectopic tachycardia can also be programmed with appropriate response to pacing to regain atrioventricular synchrony.

User profiles are built into the software and allow learners to track their performance and progress. Feedback from the system allows learners to understand if they put the patient at risk and for how long they were in unstable rhythms. The proposed simulator can be incorporated into mastery learning curricula, deliberate practice, and just in time learning. Initial experiences have been promising with 100% positive survey responses concerning the utility of the device. Users found it effective in facilitating learning pacemaker management and mimicking real-life scenarios, and all are interested in continuing to use the device to maintain and gain skills. The proposed system aims to allow learners to confidently and safely program temporary and permanent pacemakers to improve hemodynamics in any possible brady- or tachycardia. The simulator allows learners to use their actual medical equipment.

The inventors have built a prototype device that successfully simulates six training scenarios and interfaces with an OSCOR 203H external pacemaker by generating intracardiac signals and registering pacing spikes. In one embodiment, the device has a touch screen user interface that allows users to select training scenarios and customize signal parameters to run a heart rhythm simulation. During simulation, the touch screen displays ECG signals corresponding to the training scenario and pacemaker settings. The inventors have performed preliminary testing to ensure dynamic, real-time response with the pacemaker, temporal alignment of ECG and electrogram signals, as well as physiological accuracy of ECG signals and state transitions.

In an illustrative embodiment, the proposed device is fully programmable to generate a true electrical output that mimics human hearts. The educator will be able to select from any type of heart rhythm as well as specific characteristics of the rhythm of the heart, matching the variability seen in real patients. This output can be delivered to any commercially available temporary or permanent pacemaker as well as to a monitor that will display the heart rhythm. This will allow the user to use the pacemaker to assess and discover the characteristics of the virtual heart. The device is also configured to receive input from the pacemaker and respond to pacing inputs exactly as a human heart would. This will allow the user to understand the pacing characteristics of the heart. The heart will respond appropriately to the pacing and generate new rhythms affected by the pacing for display on the monitor. More advanced features such as terminating arrhythmias with the pacemaker can also be programmed.

The following discussion describes the basic components and operation of a pacemaker. Pacemakers are used in cardiology to ensure stable hemodynamic activity by providing a protective measure against arrhythmias. Specific settings of the pacemaker are tuned to the patient’s individual needs, and daily adjustments are made to ensure safe pacing of patients. Temporary pacemaker use provides the additional capability of breaking reentrant arrhythmias, a common pediatric pathology, via electrical intervention. Common reentrant arrhythmias include ventricular tachycardia, supraventricular tachycardia, and atrial flutter. Electrical intervention via the pacemaker is an established method of breaking arrhythmias. This stimulation is delivered to the atrium for most arrhythmias (a functionality called rapid atrial pacing, or RAP) and has a high probability of restabilizing hemodynamic functions.

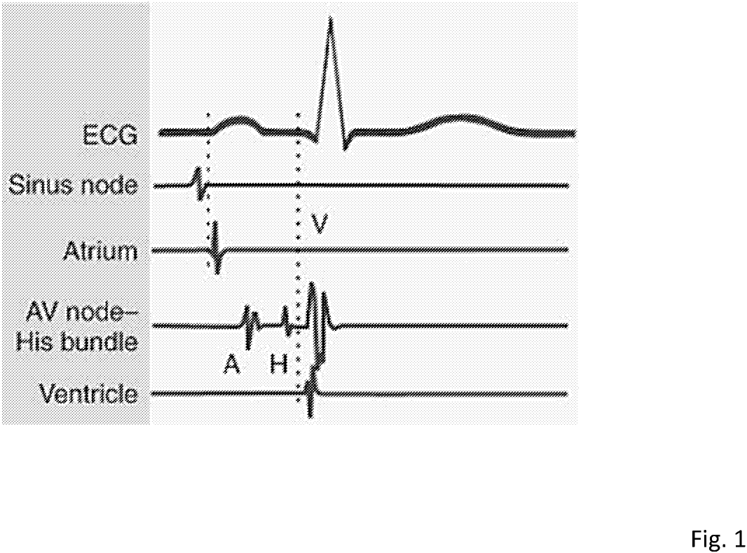

Pacemakers connect to electrodes that relay electrophysiological information to the pacemaker and facilitate pacing pulse delivery. Signals derived from these electrodes are classified as intracardiac electrograms (atrial electrograms for atrial leads and ventricular electrograms for ventricular leads). These are distinct from surface electrograms, or electrocardiograms (ECG), which contain the PQRST complex. Electrograms correspond to individual depolarizations of the atrium and ventricle, whereas ECGs capture the entire heart’s electrical activity. As a result, electrograms are temporally coordinated with the ECG signal. Atrial electrograms have small subsequent deflections corresponding to QRS complexes (i.e., combinations of Q waves, R waves, and/or S waves that represent ventricular depolarization), and ventricular electrograms have small subsequent deflections corresponding to T waves. These are noise from atrial and ventricular repolarization, respectively. Intracardiac electrograms also have 60 Hertz (Hz) background noise. FIG. 1 depicts healthy theoretical intracardiac electrograms and their temporal relation to an ECG signal in accordance with an illustrative embodiment. In FIG. 1, the signal labeled atrium is the atrial electrogram and the signal labeled ventricle is the ventricular electrogram.

To effectively induce a beat, an electrical pacing signal is used. The voltage of the pacing signal has to meet or exceed the capture threshold of the myocardial tissue. A sub-threshold voltage would fail to effectively induce a beat. The pacing spike should also occur outside of the given chamber’s refractory period (i.e., a time during which the myocardial tissue is chemically incapable of propagating electrical signals). In general, the settings of pacemakers include sensitivity threshold, output voltage, and heart rate. These settings determine the frequency and intensity of pacing spike delivery.

The sensitivity threshold refers to the minimum electrogram voltage necessary for the pacemaker to sense a heart beat. If the intrinsic heart rhythm is functioning at a normal rate, a correct sensitivity threshold will prevent unnecessary pacing. If the sensitivity threshold is too high, under sensing will occur, and the pacemaker will deliver redundant or mistimed beats. If the sensitivity threshold is too low, oversensing will occur, and background noise or heart chamber repolarizations will be detected as depolarizations, inhibiting any potentially necessary pacing. The output voltage is the pacing spike voltage, and the heart rate is the frequency of pacing spikes that are output by the pacemaker.

Pacemaker parameters are adjusted daily to ensure effective and safe pacing. Sensitivity is determined by lowering the pacing frequency to reveal the patient’s natural heart rhythm. In some implementations, the output threshold is found by slowly reducing the output voltage until the QRS complex is visibly reduced.

In the context of medicine, simulations can be described as scenarios or environments designed to closely approximate real-world situations, usually for the purposes of training or evaluation. Use of simulations have become increasingly prominent, as they provide immersive, experiential learning in a risk-free environment. Recent literature has emphasized the value of simulations used for cardiac critical care training, while analyzing future directions and challenges. The efficacy of simulation has been confirmed, and it has been shown that simulations, especially high-fidelity ones, are more effective in providing skill acquisition than any other method.

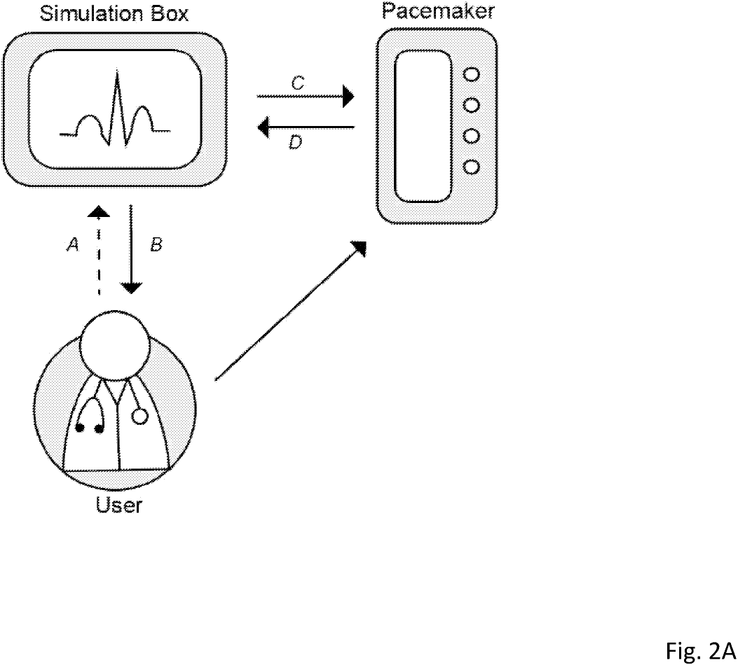

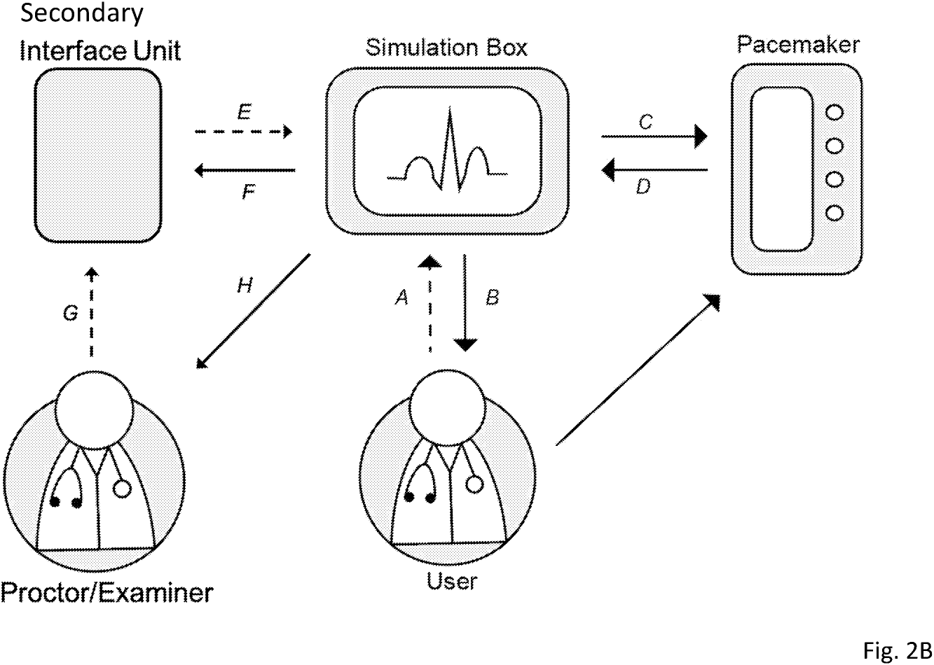

As discussed above, described herein is a standalone simulation system for training medical providers in pacemaker use. In an illustrative embodiment, a user interface of the system collects user inputs and the system generate a simulation using those inputs. In response to the inputs, the simulation system generates a digital ECG signal using a piecewise model, and electrograms are generated and processed by one or more digital-to-analog converters (DAC) for output to an pacemaker. Inputs from the pacemaker are sensed and processed by the software, as described herein. This process is described in more detail with reference to FIG. 2.

FIG. 2A is a schematic of a simulation model in accordance with an illustrative embodiment. In FIG. 2, the simulation box (or system) can contain computational components that handle the programming for all of the system functions. The computational components can include a processor, a memory, display, etc. as described in more detail below. As shown by the arrows in FIG. 2, the functions of the simulation system can include: A) receiving user input of scenario selection(s) and parameter adjustment(s); B) generating a real-time ECG signal to be displayed on the device screen; C) generating electrogram signals to send to the pacemaker; and D) processing and responding to output pacing pulses from the pacemaker. In FIG. 2, solid lines indicate interactions taking place during the simulation, and the dashed line indicates a non-simulation feature. In an illustrative embodiment, the simulation system also displays performance feedback after each simulation on the display screen.

In an illustrative embodiment, a housing of the simulation system (or box) contains the ports to be used for all connections, as well as peripheral circuitry to ensure compatibility between the pacemaker and central components. In one embodiment, the simulation system can include has a touch screen to facilitate scenario parameter adjustments and ECG signal display. The touch screen can be a light-emitting diode (LED) touch screen, a liquid crystal display (LCD) touch screen, or any other type of touch screen known in the art. In an alternative implementation, a touch screen may not be used. In such an embodiment, a keyboard, mouse, and/or microphone may be used to receive user inputs.

FIG. 2B is a schematic of a simulation model with a secondary user interface that provides an examination/proctored mode in accordance with an illustrative embodiment. The system of FIG. 2B can operate in the same fashion as the like components described with reference to FIG. 2A. However, as shown, the schematic of FIG. 2B also includes a proctor/examiner and a secondary interface unit. Although a proctor/examiner is shown as an example, it is to be understood that the secondary interface unit can be used by any user to access, monitor, and/or control the system. The secondary interface unit can be any type of computing device described herein, such as a smart phone, a tablet, a dedicated simulation device, etc. When operated in examination/proctored mode, the secondary interface unit can be used by an examiner or other user to monitor the simulation. This results in additional system interactions. For example, the proctor is able to change simulation settings on the interface unit (G), which have one- (E), or two-way (E and F) interactions with the main simulation box. The proctor will likely observe the simulation box (H), the user, and the pacemaker. The interface unit itself can be hard-wired (e.g. USB) or Bluetooth/wifi connected to the simulation box. The interface unit can be in the form of dedicated specialized hardware, or can be implemented on a user device (e.g. a cell phone with a simulation application). The user device can be in wired or wireless communication with the simulation system, depending on the implementation.

The proposed models maximize realism within the bounds of a simulation. By displaying the ECG in a similar manner to that of patient monitors, hands-on, bedside training is possible which best mimics the actual conditions for pacemaker use. This allows for the most realistic emergency and high-stress training.

Various functionalities and specifications for the proposed simulation system are described below. As discussed, the system is designed to output customizable intracardiac electrograms. In an illustrative embodiment, the analog electrograms are generated using a digital-to-analog converter (DAC). The device should be capable of generating electrograms with sensitivity thresholds within the range of the pacemaker. To this end, the expected sensitivity threshold, or voltage output from the DAC and peripheral circuitry, should match the actual sensitivity threshold, defined as the pacemaker sensitivity setting for which the pacemaker accurately senses the simulated electrogram. Following calibration, the actual sensitivity threshold should ideally be within 1% of the expected value, but other values (e.g., 2%, 3%) can be considered acceptable. These thresholds were determined experimentally based on the resolution of the pacemaker sensitivity setting and preliminary data acquired while testing.

The system is also designed to accurately measure pacing signal voltage. Pacing pulse magnitude or voltage is measured via an analog-to-digital converter (ADC). Accurate measurement of pacing voltage is critical for the simulation box to correctly respond to pacing conditions. In this context, system accuracy can be described in terms of matching the pacemaker output voltage setting to the ADC measured voltage. Functionally, only the relative measurement matters. For example, the pacemaker output setting may be 3.0 V, but the pacemaker may actually be generating 3.2 V if measured with an oscilloscope. The ADC measured voltage should be 3.0 V because the true voltage (3.2 V) is hidden from the user. After calibration, the actual or measured voltage should be within 1% of the expected voltage (the pacemaker output voltage), but other values (e.g., 2%, 4%, 5%) may be acceptable. These are preliminary thresholds based on initial data from testing.

The simulation system is also designed to respond dynamically and in real time to the pacing signal. Such dynamic, real-time system response to pacing conditions is important for effective, high-fidelity simulation. When a pacing pulse is received, a block of code can be executed to determine how the simulated ECG signal should change. Execution of the code includes recognition of the full range of pacing spike amplitudes, and the corresponding response calculations. In an illustrative embodiment, this code should take no longer than 0.017 s (60 Hz) to execute because that is the update rate of the touch screen. In other words, the next ECG value should be determined before the screen refreshes with a new frame. In some embodiments, an acceptable threshold of 0.034 s would allow a maximum of one new frame to be displayed on the screen without an updated ECG. In alternative embodiments, a different screen refresh rate may be used (e.g., 120 Hz, 240 Hz, etc.), which results in shortened execution times for the code that determines a response to the pacing signal.

The simulation system is also designed to have temporally aligned ECG and electrogram signals. The simulated ECG and electrogram should align in time with minimal delay between the two for real-time simulation. For the same reasons discussed above with respect to the dynamic response to the pacing signal, the delay to temporally align the signals should ideally be less than 0.017 s, but 0.034 s can be acceptable for the same reasons discussed above. In alternative embodiments, a different screen refresh rate may be used, which results in different execution times for the code that temporally aligns the signals.

The simulation system is designed to present physiologically realistic simulations, which can be based on decision trees that maintain the ECG in the appropriate state based on input conditions. The displayed ECG on the touch screen can be a ‘Lead I’ ECG, in some embodiments, and the default signal parameters for each ECG scenario mimic physiological values. Noise and parameter variation can also be included to emulate a real ECG signal. Beat-to-beat heart rate and ECG component amplitude can be updated for every cycle, and random electrical noise can also be added to the signal to achieve a realistic ECG. PR and QT intervals can scale linearly with heart rate, or length of the beat cycle.

The system also includes a navigable user interface in which signal parameters can be customized, user performance data can be stored and retrieved, and user error is taken into consideration. In some embodiments, the user interface (UI) is implemented with a graphical touch screen application. The application can enable users to customize simulations by setting ECG and electrogram parameters and choosing training scenarios. These user inputs can be stored and sent from the main processor (e.g., a Raspberry Pi or other processing component) to a controller (e.g., a Teensy or other processing component) without error so that the simulation corresponds to what the user intended.

User performance data is tracked by the system. The user performance data refers to information summarizing a particular user’s actions during each training scenario, as well as overall statistics on their proficiency with proper pacemaker usage. This data may include an amount of time taken to resolve a scenario, a number of inappropriate actions made during a scenario, a number of scenarios for which the user is considered “well-trained,” a number of errors made by the user, etc. Ensuring that performance data is accurate during retrieval and storage is critical for the device performance and users’ learning. Additionally, every ECG and electrogram parameter that the user can set may have a range and resolution that is limited by either physiology or device hardware. To prevent users from inputting invalid values, the UI should limit user input to valid ranges and increments and notify users what values are allowed. Accounting for user errors improves the usability of the device and facilitates smooth running of simulations.

The proposed system can also be designed to be compatible with all pacemaker models. To achieve this, the device can use 2 millimeter (mm) banana plug connections which are compatible with available pacemaker models. In alternative embodiments, different connection types/sizes may be used depending on pacemaker model.

Lastly, the proposed system should be intuitive for the user/proctor to use. Specifically, a well-designed UI should be intuitive to its intended users, who should be able to use it to perform tasks without being guided through step-by-step. Primary users will be medical fellows and cardiology staff. Ease of use is important for maximizing training efficiency and learning outcomes. In one embodiment, the system has target and acceptable thresholds of <1 min and <2 min for the user to set up the simulation, while the system has target and acceptable thresholds of <2 min and <3 min for the user to create a user profile. These are arbitrary thresholds chosen to benchmark the initial testing, and alternative thresholds may be used as the system is used more frequently.

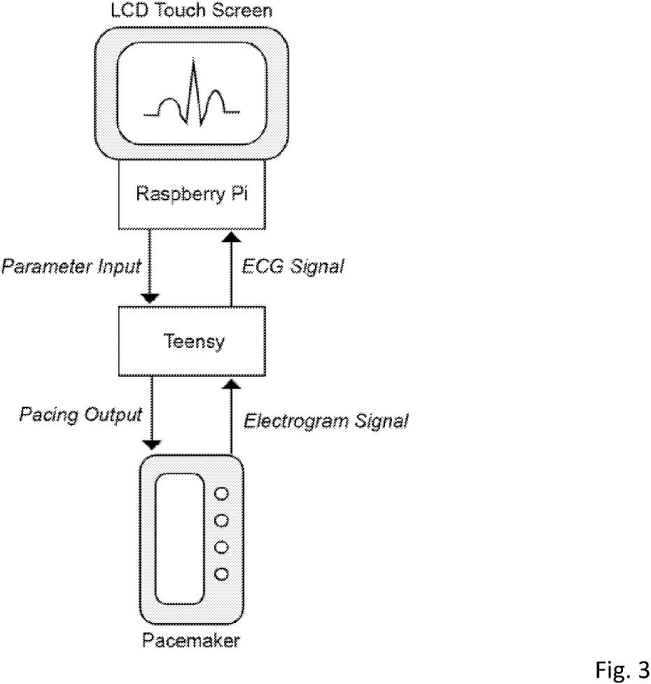

FIG. 3 is a block diagram depicting a design scheme for the simulation system in the form of a closed-loop system in accordance with an illustrative embodiment. In the depicted embodiment, the simulation system includes two subsystems: a main processor in the form of a Raspberry Pi (RPI) connected to a 7 inch LCD touch screen and a controller in the form of a Teensy microcontroller development board. As discussed below, different hardware components can be used in alternative embodiments. The RPI can be used to manage the UI application and send user inputs, in the form of signal parameters, to the Teensy. The Teensy can send the simulated ECG signal to the RPI via USB serial data transfer (or other method) for display on the display screen. The Teensy can also output time-correspondent intracardiac electrograms to the pacemaker using a digital-to-analog converter (DAC). The pacemaker will sense the electrograms and either send or not send pacing output back to the Teensy depending on its parameter settings. This pacing output will trigger an external interrupt allowing the Teensy to recognize pacing output receipt. Pacing output amplitude (voltage) will be quantified using an analog-to-digital converter (ADC). Based on the nature of the sensed output, the Teensy will alter the ECG and electrograms following decision trees, which are described in more detail below.

In the embodiment of FIG. 3, the subsystem responsible for signal simulation/generation is a PJRC Teensy 3.6. It was chosen for its advanced processing power and speed, flexibility, and strong analog capabilities. The key technical specifications for the Teensy 3.6 include a 180 MHz ARM Cortex-M4 processor with a floating-point unit for fast floating-point calculations, built-in DACs and ADCs for analog signal processes, and numerous external interrupt enabled pins to recognize pacing input.

The subsystem responsible for the UI and ECG signal display is a Raspberry Pi 4B connected to a Raspberry Pi 7″ capacitive touch screen. The 4B model is the latest version and has the greatest amount of random access memory (RAM) (4GB), which is important for smooth running of graphical user interfaces. A touch screen is used because it allows the ECG display and user interactions to be serviced by the same hardware and a single application (as opposed to having a screen for display and other hardware for user inputs). This particular touch screen was chosen because it is the official version which means it is well-documented and has good technical support from online help forums. While the RPI is capable of handling the responsibilities of both subsystems by itself, the Teensy was included to produce a prototype. In the embodiments described herein, the system can utilize a Teensy, an RPI, and/or a different processing component.

While the embodiment of FIG. 3 depicts use of a Raspberry Pi and a Teensy device, it is to be understood that the system is not so limited and that any other computing devices (processors, memories, transceivers, etc.) may be used to perform the processing and communication/control functions of the components depicted in FIG. 3. Similarly, while a touch screen is described herein as a user interface, the user interface may be different in other implementations and may include a keyboard, mouse, microphone, a standard (non-touchscreen) visual display, etc.

In an illustrative embodiment, the system can be implemented in a housing that incorporates one or more connections to the pacemaker, a power source, and one or more USB ports for software updates and communication. The system circuitry and/or components can be translated into a PCB to minimize physical footprint and integrate into the housing. The housing design can be formed to maximize durability and ergonomic use. This includes incorporating a handle, stand, screen protector, rubber cushions, etc.

The circuit interface between the Teensy and the pacemaker is referred to as the pacemaker circuit. In one embodiment, the pacemaker is the OSCOR 203H pacemaker model, although different pacemakers may be used in alternative embodiments. The output from this circuit feeds into two more circuits: a sensing circuit and a measurement circuit for sensing and measuring an incoming pacing spike, respectively.

The range of pacemaker voltage output (0.1-18.0 V) poses two problems. First, a minimum voltage of ~3.0 V is required to trip the external interrupt. Second, no pins should receive a voltage greater than 3.3 V (i.e., any input greater than 3.3 V will short the Teensy). The sensing circuit used addresses this problem in two stages. First, the output from the pacemaker circuit is divided by six and flipped using an inverted op amp to prevent receipt of a pacing pulse greater than 3.3 V. A specialized op amp with overvoltage protection is used. The output from this stage feeds into a noninverting op amp that rectifies and amplifies the signal by 200 so the output will always be 3.3 V even at the lowest pacing output setting (0.1 V at the input of the sensing circuit, 0.0167 V after the first stage). The output from this circuit is connected to the Teensy interrupt pin for pacing pulse sensing.

The two-stage measurement circuit shares its first stage with the sensing circuit. The second stage rectifies the signal using a unity buffer and feeds into the ADC pin on the Teensy for pacing pulse measurement. Additionally, one set of peripheral circuitry is implemented for each chamber of the heat (atrium and ventricle) and is connected to the appropriate terminals on the pacemaker. In one embodiment, the entire system can be integrated into a custom-designed printed circuit board (PCB). The PCB will connect to the pacemaker via 2 mm banana plugs, which are standard connectors for pacemakers.

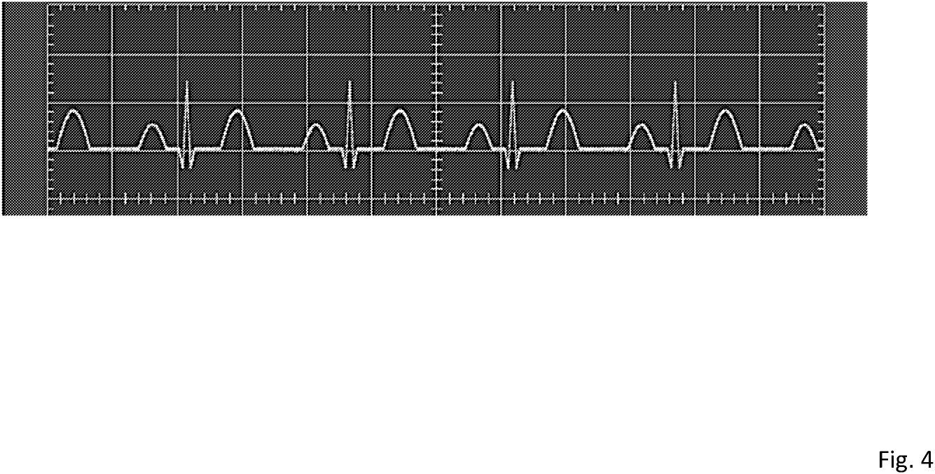

The simulated ECG signal is generated using a piecewise technique that generates each individual component of the signal sequentially (e.g., first the P wave, second the QRS complex, third the T wave). The origins for each piecewise element can be simple geometric shapes (straight lines, held values, parts of sinusoids, etc.), or taken from digitized real ECG signals which have been broken down into a library of piecewise elements. In some embodiments, additional (but optional) simulated signal complexity can be chosen to include high-frequency signal noise and/or low-frequency signal drift/baseline undulations to mimic real world situations. Similarly, electrograms can optionally include a randomized component of their impulse amplitudes. FIG. 4 depicts a simulated normal sinus rhythm ECG signal at 120 beats per minute (bpm) in accordance with an illustrative embodiment. Established ECG parameters are used to model each waveform component to achieve a realistic signal. The main advantages of this method are ease of varying signal parameters and intuitive implementation.

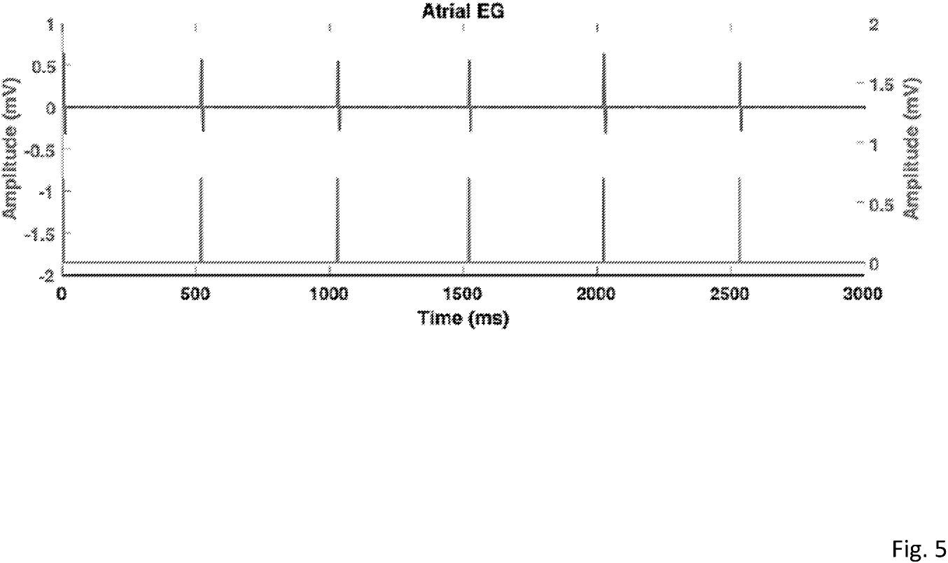

In an illustrative embodiment, each ECG simulation has an underlying state, designated by user input, and numerous possible states that it can transition to via pacing input. Signal generation code was implemented using object-oriented programming in C++, although a different programming language may be used in alternative embodiments. Different heart rhythms were separated into classes, all derived from a base class that encodes normal sinus rhythm. One function varies signal parameters (e.g., heart rate and ECG component amplitudes), and another generates a single ECG cycle that sequentially produces all ECG components, as shown in FIG. 4. Component functions generate the ECG signal, using the piecewise technique described herein, and time-coordinated electrograms. Electrograms are simulated as impulses for simplicity. FIG. 5 depicts a realistic electrogram (top) and a simulated impulse electrogram (bottom) plotted in MATLAB to visualize differences in shape in accordance with an illustrative embodiment. Simulated electrograms here are idealized. A device’s actual electrograms will be noisier, but not enough to interfere with pacemaker sensing.

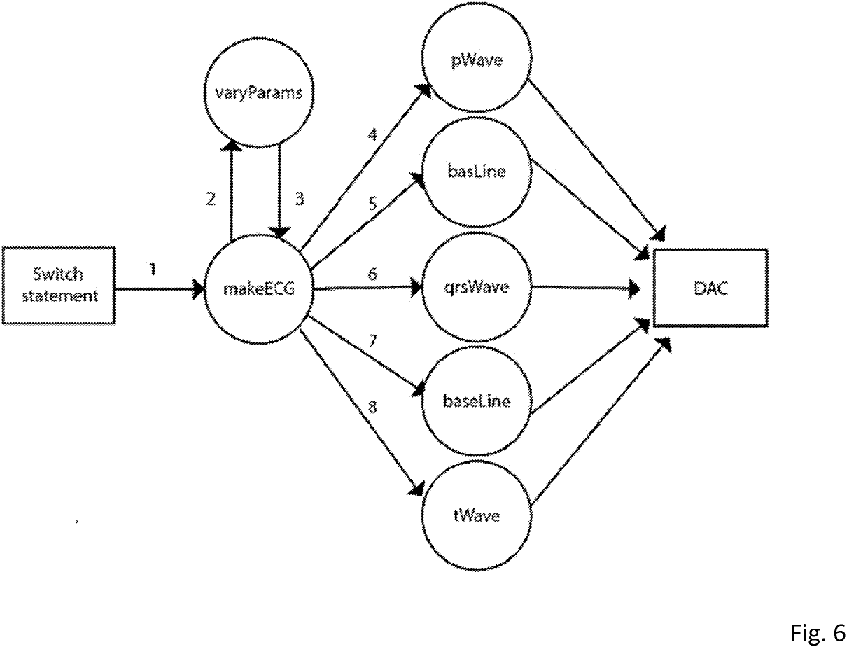

It is noted that the lack of downward deflection present in real electrograms does not impede sensing by the pacemaker. In the component functions, electrogram signals are sent to the DAC and ECG signals are sent to the RPI via a USB serial data transfer. FIG. 6 depicts an ECG generation code workflow in accordance with an illustrative embodiment. Interrupt functions facilitate asynchronous response to pacing spikes from the pacemaker. Based on the amplitude of the pacing spike and the time index of the ECG cycle at which the pacing spike occurs, the interrupt function determines how to alter the ECG (if at all) using sequential if-else statements based on the decision trees.

In an illustrative embodiment, training scenarios for the proposed system are fully developed simulations, both for daily adjustments and emergency situations, that comprehensively test trainee knowledge of pacemaker use. Training scenarios are defined as an underlying heart rhythm with a corresponding ECG, corresponding electrograms, the states that the ECG can adopt, and how the ECG can transition between those states. The intended use is to proctor simulated pacing assessments using the device. While there will be scenarios with preset heart rates, sensitivity thresholds, and capture thresholds, the proctor will be able to customize these parameters to create a unique training experience. Each scenario will have a decision tree that determines how changing the three pacemaker settings alters the simulated ECG. The decision trees are developed based on research and user feedback. Although comprehensive training is important, teaching the basic concepts of pacing (e.g., finding the sensitivity and capture thresholds) is also a high priority learning outcome.

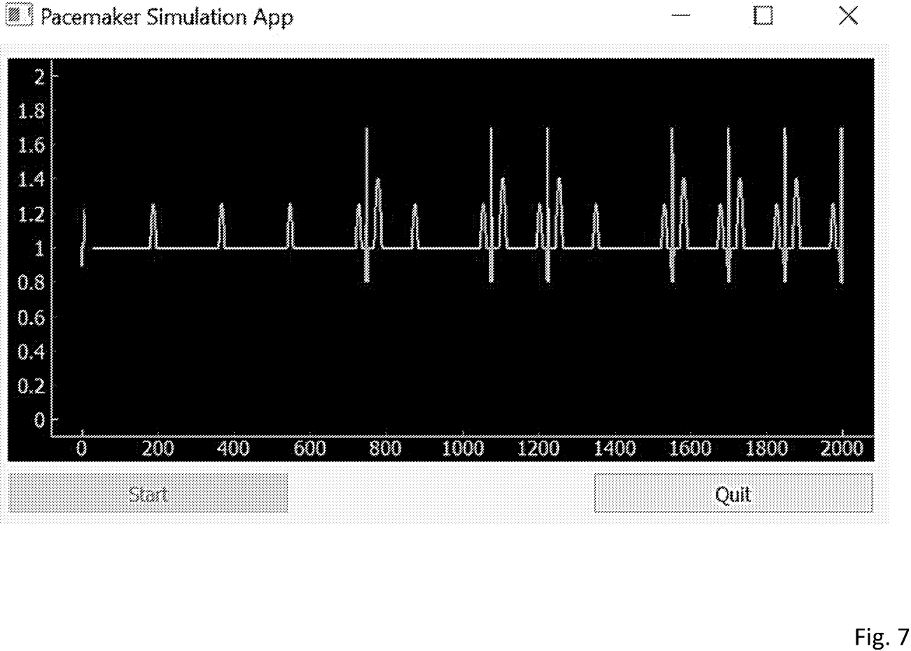

The user interface (UI) can include the RPI, LCD touch screen, USB connections to the Teensy, and a UI application. As used herein, user interface can refer to the overall hardware and software subsystem that facilitates user interaction with the simulation. The UI application refers to the software that the RPI or other processing component runs. The RPI is responsible for running the UI application, which displays the user-facing graphical interface to the touch screen and performs backend functions such as USB communication. The UI application allows users to pick training scenarios and set simulation parameters. When the simulation begins, the RPI reads ECG values from the Teensy and displays them on the touch screen. FIG. 7 depicts an ECG displayed in a user interface application during a simulation in accordance with an illustrative embodiment. Specifically, the example in FIG. 7 shows a Mobitz Type II heart block training scenario.

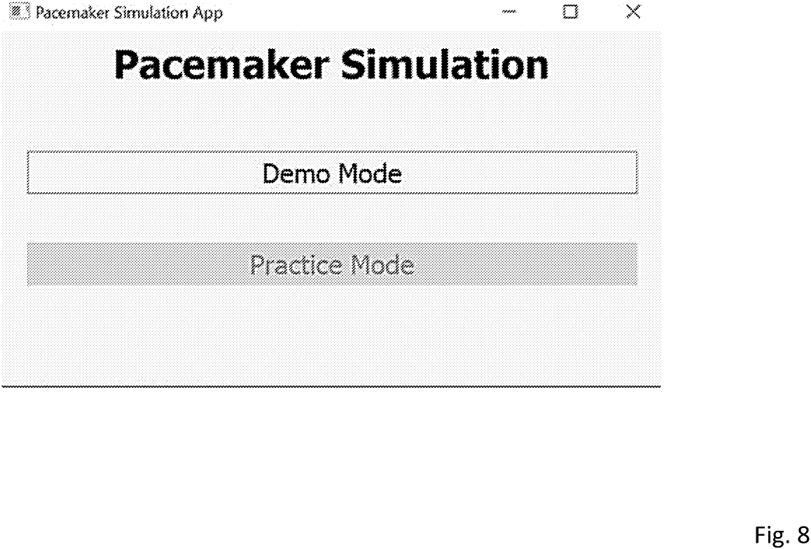

In some embodiments, there are two main use cases for the system, resulting in two modes of operation for the application. In alternative embodiments, additional operational modes may be included. FIG. 8 depicts a user interface application screen shot of operational modes in accordance with an illustrative embodiment. As shown in FIG. 8, these are the Demo and Practice modes displayed on the Main Menu page, which can be the first page that the user sees when the application is run. Demo mode will primarily be used by instructors or expert users, who would select specific training scenarios, set ECG and EG parameter values for that scenario, and then demonstrate proper pacing techniques to medical trainees using the simulation. Practice mode will primarily be used by medical trainees to get more hands-on practice with pacing. This mode generates a random scenario with randomized parameters for practice. Users will be informed of the underlying state of the scenario when they quit the simulation. The system can also include a third Proctored (or Examination) mode in some embodiments. In the Proctored mode, the proctor is able to set up a specific scenario for the user (i.e., trainee) to handle. Alternatively, the proctoring aspects of the system can be incorporated into the Demo mode.

A universal service bus (USB) connection was used to communicate between the Teensy and RPI because USB cable is already used to power the Teensy from the RPI and because the protocol is relatively straightforward to use. To make the ECG plot more realistic looking, a background and green line were used to mimic the look of ECG displays on hospital monitors. Like a real ECG display, the plot loops over itself when it has reached the end of the horizontal axis. Additional details on the software implementation are described below.

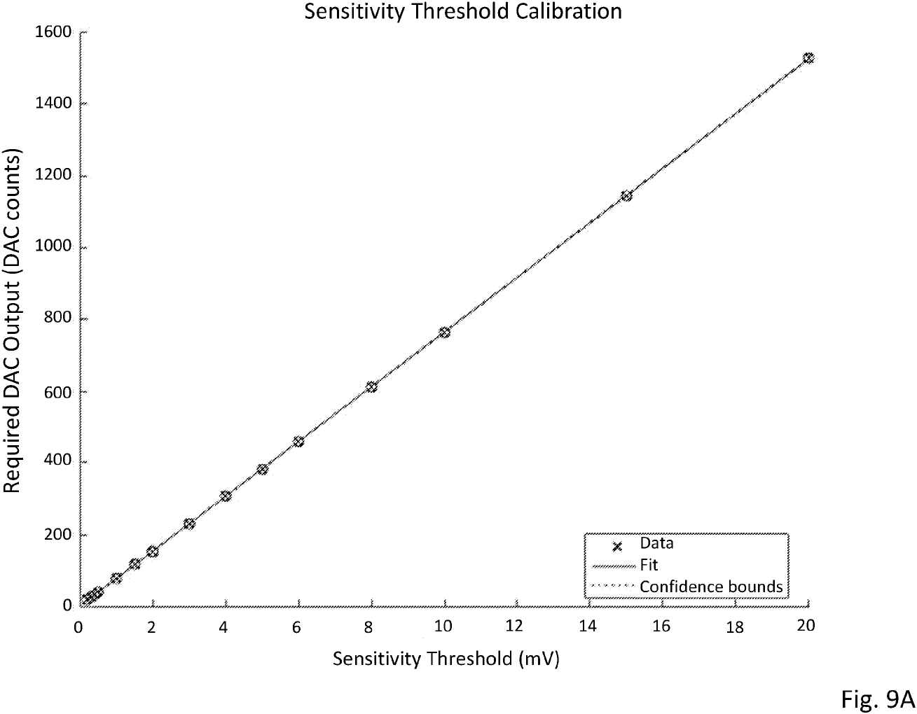

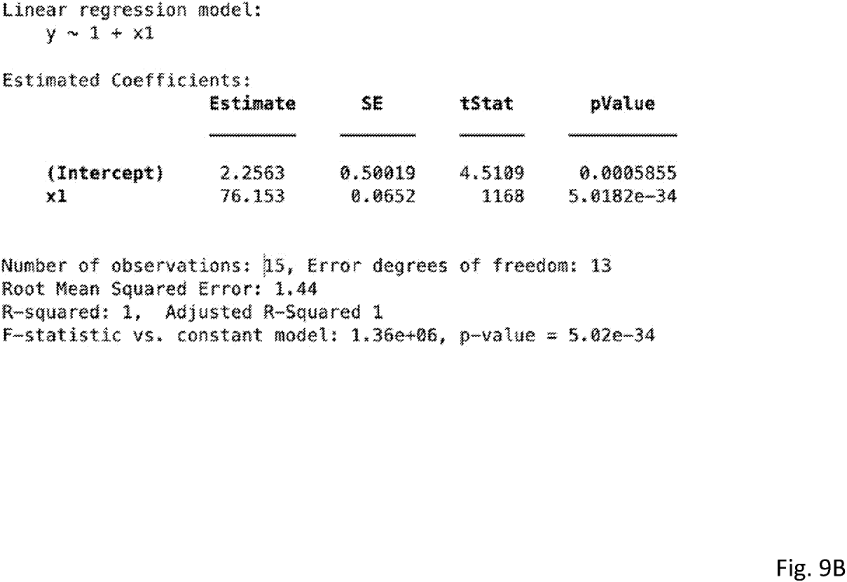

To determine the relationship between required electrogram sensitivity threshold (or required DAC value to generate a sensed electrogram) and the pacemaker sensitivity setting, peripheral circuitry was set up and connected to an OSCOR 203H pacemaker and the Teensy. The Teensy was programmed to output an electrogram at a frequency of 2 Hz using the built-in DAC. Four push buttons were wired to the Teensy; two of these altered the amplitude of the electrogram by +/- 0.806 mV, and two altered the amplitude by +/-8.06 mV. Push buttons were used to change the amplitude of the electrogram to avoid rewriting and uploading code to the Teensy every time it was desired to change the electrogram amplitude. The pacemaker sensitivity setting was set to the lowest value (0.2 mV), and the buttons were used to adjust the electrogram amplitude until the pacemaker sensed the electrogram consistently at which point both the pacemaker sensitivity setting and the electrogram amplitude in DAC counts were recorded. The pacemaker sensitivity setting was increased to the next value and the process was repeated. At least 20 consecutive sensing events without pacing were conducted. Pacemaker sensitivity setting has a range of discrete (not continuous) values. FIG. 9A depicts how a linear model was fitted to the data to determine the relationship between required DAC output and pacemaker sensitivity setting in accordance with an illustrative embodiment. FIG. 9B is a table depicting data corresponding to the linear model of FIG. 9A in accordance with an illustrative embodiment.

The linear fit generated the following equation relating the DAC output to pacemaker-measured sensitivity threshold:

$$r e q u i r e d D A C o u t p u t = s e n s i t i v i t y t h r e s h o l d * 76.153 + 2.256$$

The R² value (1) indicates a perfectly linear fit, and the low p-values for the intercept and slope (both <0.05) indicate the equation is a good fit for the data. Power was not calculated, but the clear linear relationship suggests it may not be necessary. This equation was implemented into the project code library to convert a desired sensitivity threshold to a matching analog output.

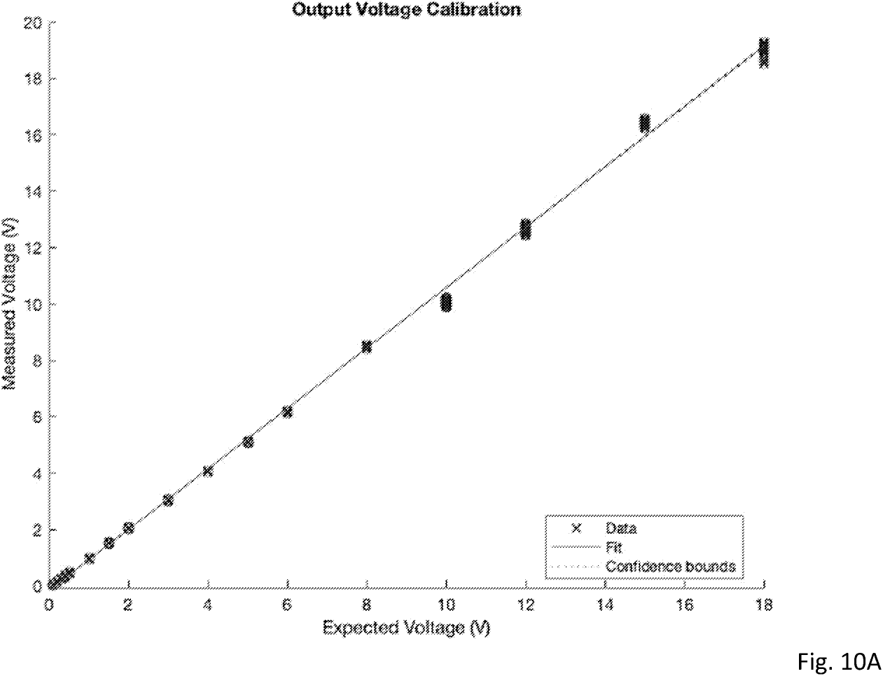

The same setup was used to accurately measure pacing pulse amplitude or voltage. The Teensy was set to not output an electrogram signal so the pacemaker would only pace (never sense). Every time a pacing pulse was sent from the pacemaker, it was measured using the ADC on the Teensy. The measured voltage was converted from ADC counts to volts using the resolution of the ADC and the voltage division ratio in a measurement circuit and recorded as the actual voltage. Resolution = V_{ref}/(2ⁿ-1) where V_{ref} is 3.3 V and n is the 13 bit resolution such that the resolution is 0.40 mV. At least 30 data points (pacing pulses) were measured for each trial or pacemaker output voltage setting. The pacemaker output voltage setting was recorded as the expected voltage. This process was repeated for the full range of voltages (0.1-18.0 V). FIG. 10A depicts how a linear model was fitted to the data to determine the relationship between actual and expected voltages in accordance with an illustrative embodiment. FIG. 10B is a table depicting data corresponding to the linear model of FIG. 10A in accordance with an illustrative embodiment.

The linear fit generated the following equation relating the measured voltage to the expected voltage:

$$m e a s u r e d v o l t a g e = e x p e c t e d v o l t a g e ∗ 1.071 - 0.130$$

The high R² value (0.999) confirms a linear fit is appropriate, and significant p-values for the intercept and slope (<0.05 for both) indicate that the linear model is an accurate fit for the data. Again, power was not calculated, but the clear linear relationship suggests it may not be necessary. This equation was incorporated into the project code library to correct for the difference between the ADC measured voltage and the pacemaker setting.

To verify that the displayed ECG signal meets the users’ standard of accuracy in order to preserve the efficacy of the simulation, screenshots of the ECG signal were taken for the following five rhythms: normal sinus rhythm, sinus bradycardia, heart block type I, JET, and atrial flutter. The users stated that rhythms met their standard of physiological accuracy. Additionally, to determine that the system responses to pacing input reflect physiological responses, decision trees were verified with the system users. Sinus bradycardia and heart block scenarios were presented and walked through with user input.

Testing of the proposed simulation system focused on two major areas. First, appropriate functioning of the simulation was verified. System processing of pacing spikes should happen quickly enough to not impede the simulation. Also, system responses to pacing spikes, including state transitions, should reflect the logic illustrated in the decision trees. The ECG signal and the electrogram signals are also to remain temporally coordinated through display and pacemaker recognition, respectively. Second, the UI has been thoroughly tested for efficiency and errors. User testing was conducted to ensure that users are able to intuitively and efficiently navigate the UI. Tests were also run to ensure the efficacy of individual UI functions, including storing and transferring user inputs, storing and displaying user performance data, and appropriately displaying error messages.

The failure modes of the system have also been identified, and corrective controls for each failure mode are implemented as part of the system. The highest risk priority for signal conduction is cable/connector port damage. This can arise from normal wear or dropping the device, etc. Such port damage interrupts device-pacemaker communication until the damaged component is replaced. To control for this, the user is provided with multiple sets of cables, and the box (device) will be ergonomic so it resists slipping. Corrective actions can include switching to high gauge wires and recessing the connection terminals to reduce the chance of cable/connection port damage.

With respect to the user interface, the most critical UI failure modes include frozen menus, unexpected user actions, touchscreen response failure, and user profile corruption. Most of these arise from code errors with the exception of the touchscreen response failure, which can also result from dropping the device. These failure modes can have varying levels of severity, ranging from clicking an extra button to making the device inoperable. The current controls for code errors is extensive testing of each user interface option. Additionally, the user can reset the device if they encounter such an error. The corrective action that addresses the software errors will be providing software updates after a user identifies an issue.

A highest priority failure mode for circuit-pacemaker interfacing is circuit failure. Mechanisms of failure include individual component failure, device shorting, high current draw, and dropping the device. All of these mechanisms can render the device inoperable. Implemented current controls include choosing appropriately rated components, using overvoltage-protected op-amps, and ensuring current draw is within a safe threshold. In one embodiment, all the central and peripheral circuitry will be contained in a single printed circuit board (PCB) to reduce the chance that components are dislodged during device use. Corrective actions include providing an estimated device lifetime, shutting down the device if electrical operating conditions become dangerous, and encasing the device in an ergonomic, protective layer.

There are also two main failure modes in the dynamic response of the device to pacemaker input. The first failure mode is that the ECG responds in real time, but is not physiologically accurate, affecting the quality of performance of the device. This is caused by code errors or physiological scenarios that were not considered in the design and results in an inaccurate simulation that does not appropriately respond to user input. Current controls include testing of every foreseeable condition and using user feedback as expert opinion on the physiological accuracy of the simulations. The corrective action can be to provide users with a user manual that denotes all conditions implemented in the code. The second main failure mode is that the pacemaker input has a delayed visual response on the screen. There are several potential causes for this complication such as inefficient code on the Raspberry Pi (or other processing component), inefficient code on the Teensy (or other communication component), or a memory leak. This failure mode would also create an unrealistic simulation that results in a poor user experience and learning outcomes. The current control is testing to verify that the delay from signal to visualization is not significant. Future corrective actions would include software updates if users deem the response as too slow.

In one embodiment, the proposed simulation system can be configured to provide users with Atrial/Ventrical (AV) dissociated simulation scenarios that necessitate separate pacing responses for the atrium and the ventricle, as well as ECG generation code that allows for real-time addition of different components. Because of this, the interrupt code can be modified to account for atrial and ventricular responses and a new class of rhythms can be used that allow for ECG components to be added.

Additionally, the parameters to be stored and displayed by the system for the best learning experience possible can be finalized based on feedback. These parameters include but are not limited to: total time to resolve arrhythmia scenario, number of events in which patient is placed at risk, and pass or fail indications. Users can be consulted to determine which parameters are ultimately of the most importance.

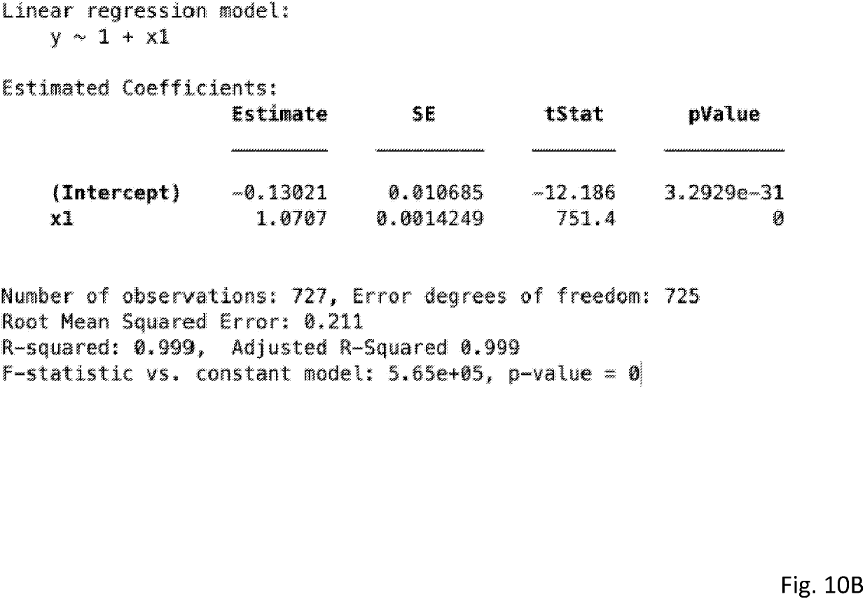

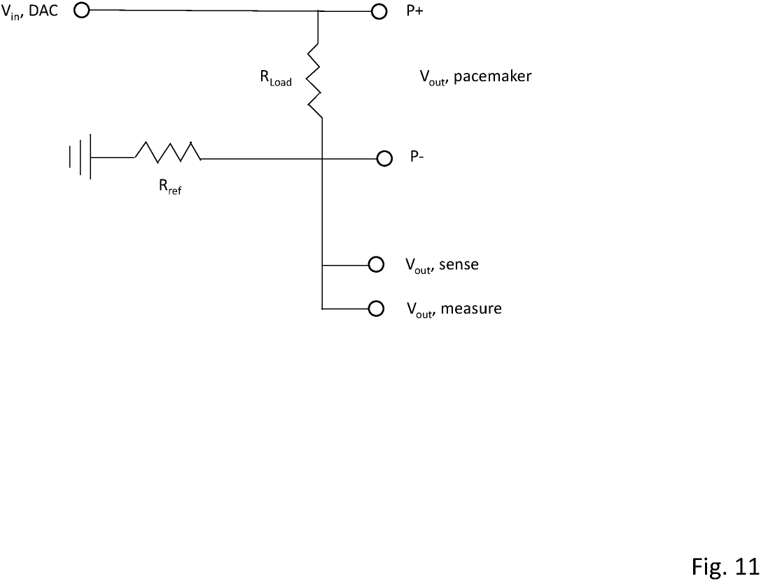

Details of the system hardware and software are provided below, with reference to the figures. In an illustrative embodiment, the system includes a pacemaker circuit. FIG. 11 depicts a pacemaker circuit in accordance with an illustrative embodiment. The pacemaker circuit interfaces the pacemaker with the Teensy, or other communication/processing component connected to the pacemaker. A load resistor, R_{load} (330 Ω), simulates the impedance of the heart, and its value was chosen based on the load impedance range listed in the OSCOR 203H manual. A series resistor to ground, R_{ref}(33 kΩ), establishes the reference voltage, and its value was chosen experimentally so that the entire pacemaker sensitivity threshold range (0.1-20.0 mV) can be accounted for with the resolution of the DAC. Resolution = V_{ref}/(2ⁿ-1) where V_{ref} is 3.3 V and n is the 12 bit resolution so the resolution is 0.80 mV. Together, these resistors function as a voltage divider for the simulated electrogram, V_{in,DAC}. In alternative embodiments, different resistor values and/or a different pacemaker circuit configuration may be used.

Given that the pacemaker senses a differential voltage, V_{out,pacemaker}, across R_{load}, an analytical relationship between the voltage of the simulated electrogram V_{in,DAC} and the expected sensitivity threshold V_{out,pacemaker} can be determined where Rᵢₙ (22kΩ) is the input impedance of the pacemaker:

$${V}_{o u t} = {V}_{i n} \left(1 - \frac{{R}_{r e f}}{{R}_{r e f} + {R}_{l o a d} / \text{} / {R}_{i n}}\right)$$

This relationship does not hold true when testing sensitivity threshold. As discussed above, an experimental relationship was determined to characterize sensitivity threshold. Still referring to FIG. 11, the value V_{out,sense} feeds into a sensing circuit, and V_{out,measure} feeds into a measurement circuit.

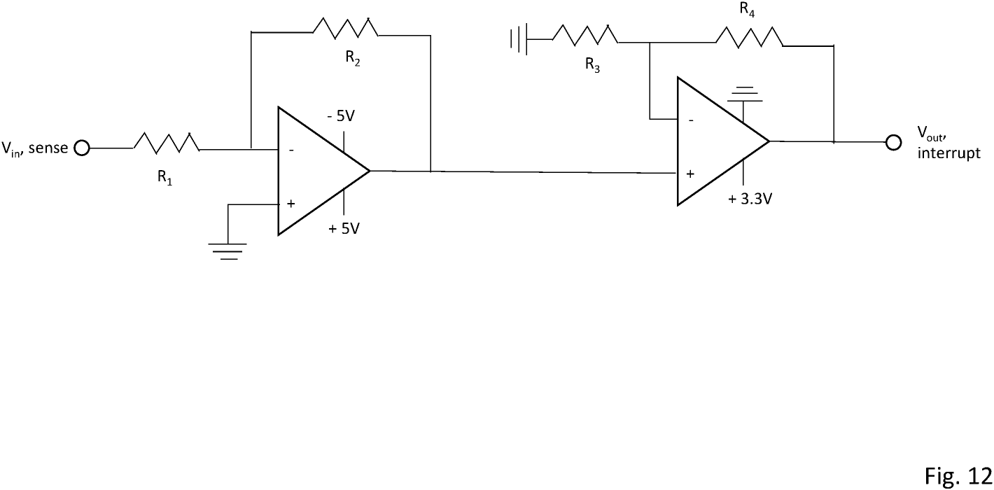

FIG. 12 depicts a sensing circuit in accordance with an illustrative embodiment. The sensing circuit is designed to safely trigger an external interrupt pin by turning the negative pulses from the pacemaker into positive 3.3 V impulses. The first stage of the sensing circuit divides and flips the pacing pulse so it never exceeds 3.3 V to avoid shorting the Teensy. A specialized op-amp with overvoltage protection (ADA4177) allows safe input of voltages up to 32 V greater than and less than the op-amp’s power supply voltages. The ADA4177 is a dual-supply op-amp specified at +/-5 V [29]. The +5 V is sourced from the RPI, and the -5 V is generated using a switched-capacitor inverting charge pump (MAX660). The MAX660 output voltage is not regulated, but the load current, or supply current required for the ADA4177 (1 mA), is significantly smaller than the specified load current for noticeable voltage dropout (100 mA). For a +5 V source, 100 mA current draw results in a voltage dropout of ~0.43 V, which is estimated from an output voltage drop vs. load current graph. The second stage of the sensing circuit amplifies the signal by 200 so that the minimum V_{in,sense} (0.1 V) will always reach 3.3 V at V_{out,interrupt}. The now 3.3 V pulse signal can consistently trigger the external interrupt. In one embodiment of the sensing circuit, R₁ = 600 kΩ, R₂ = 100 kΩ, R₃ = 10 kΩ, and R₄ = 2 MΩ. In alternative embodiments, different resistor values may be used and/or different circuit components can be used to implement the sensing circuit.

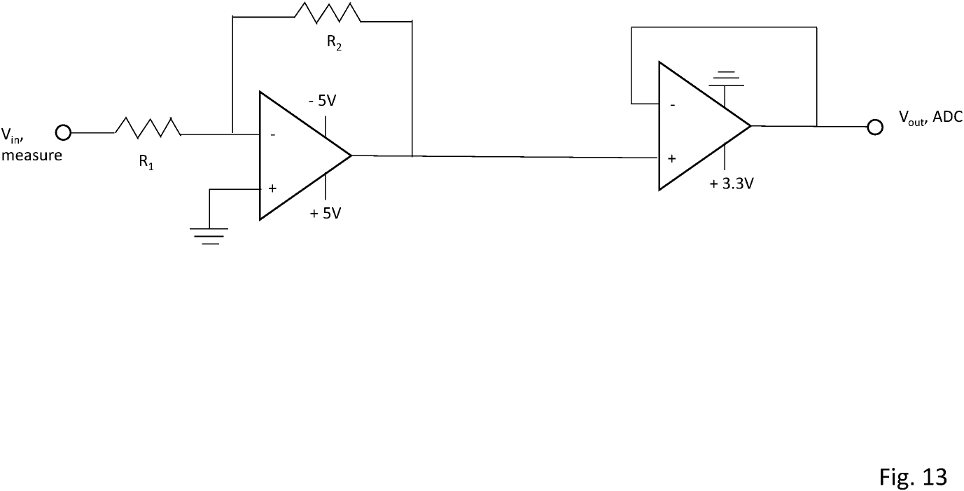

FIG. 13 depicts a measurement circuit of the system in accordance with an illustrative embodiment. The measurement circuit enables safe measurement of pacing pulse voltage. It shares its first stage with the sensing circuit to protect against voltage inputs greater than 3.3 V. The signal is then rectified and sent to the ADC pin to read the voltage. Internally, the measured voltage is multiplied by six to account for the voltage division that occurs in the first stage. Therefore, the expected voltage should be ⅙ of the actual voltage. In practice, this generally holds true but there can be high variation in the measured voltage. The exact relationship was quantified experimentally as described above. In one embodiment of the measurement circuit, R₁ = 600 kΩ and R₂ = 100 kΩ. In alternative embodiments, different resistor values may be used and/or different circuit components can be used to implement the measurement circuit.

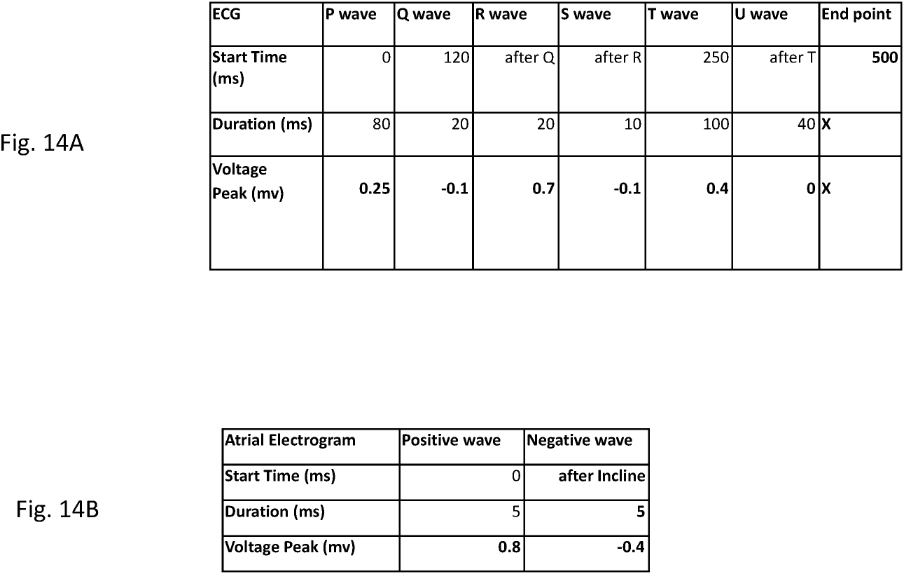

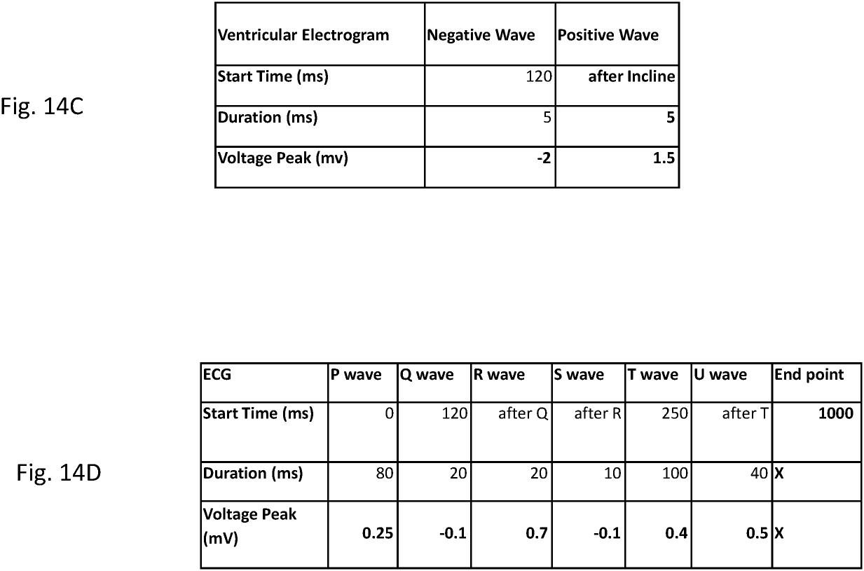

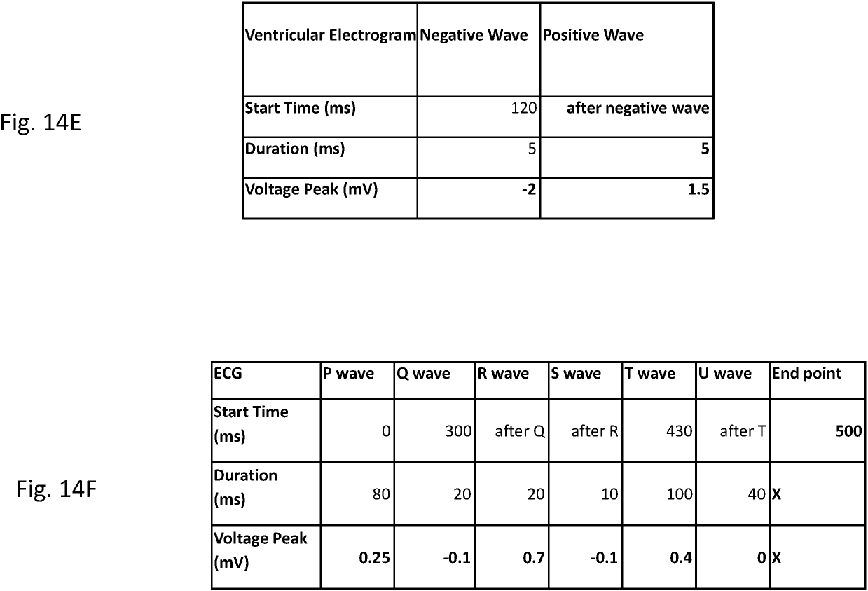

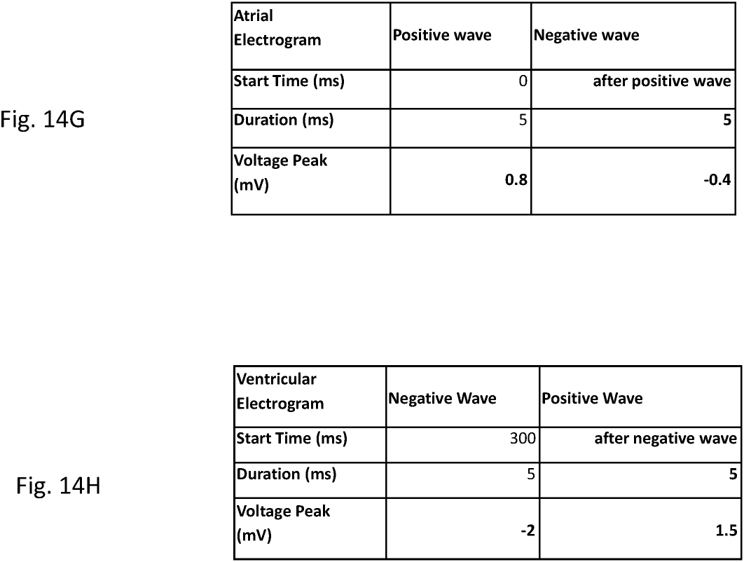

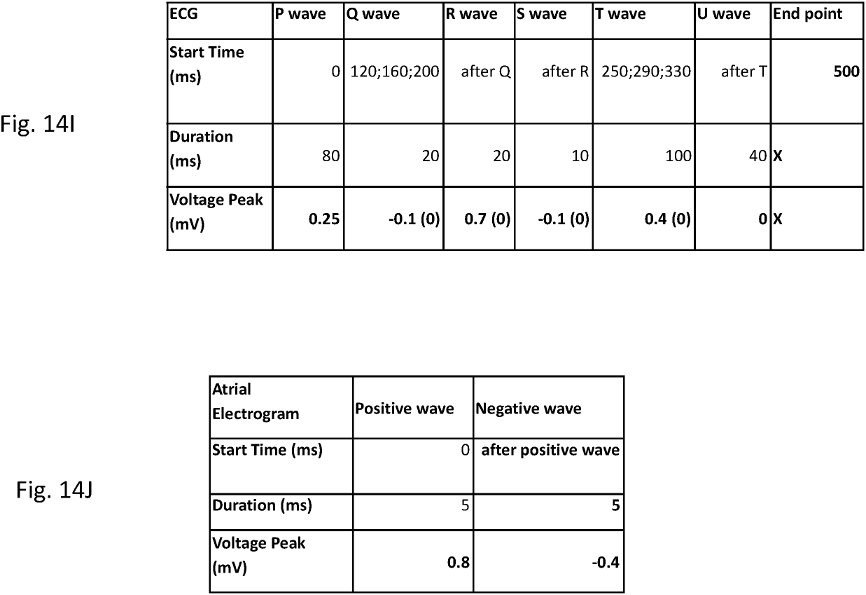

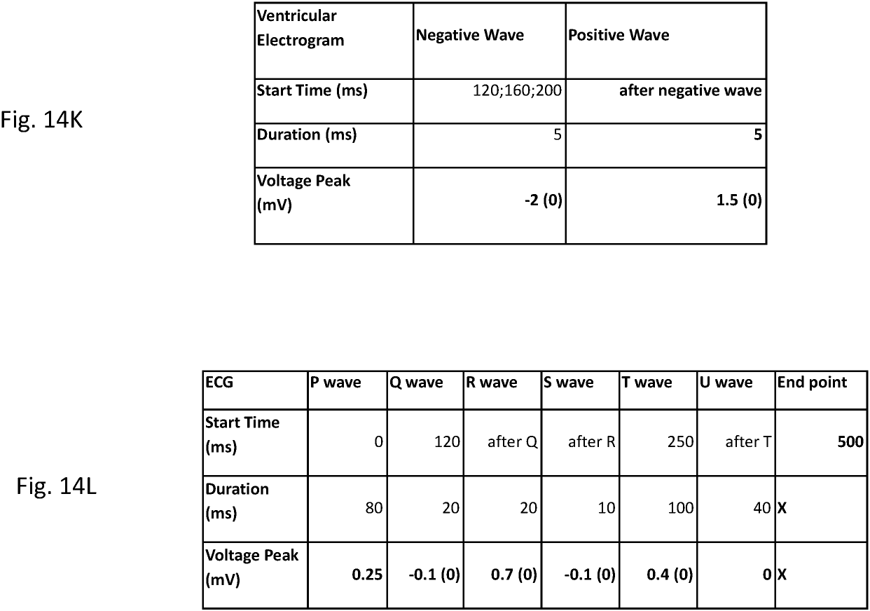

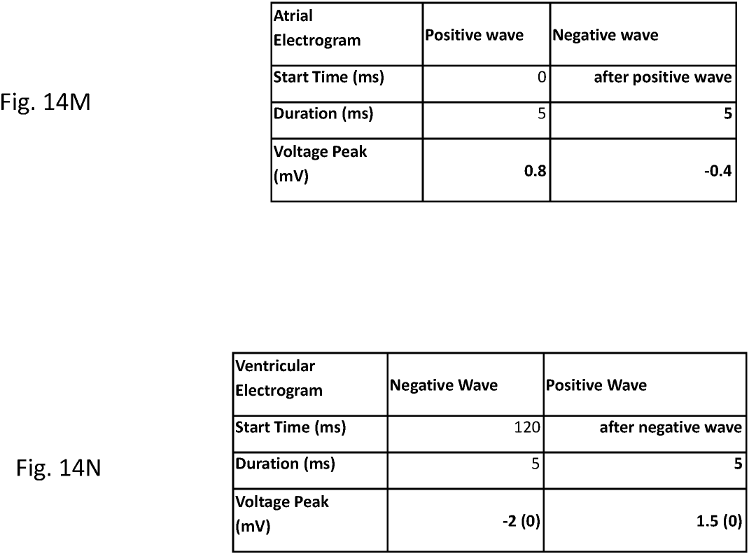

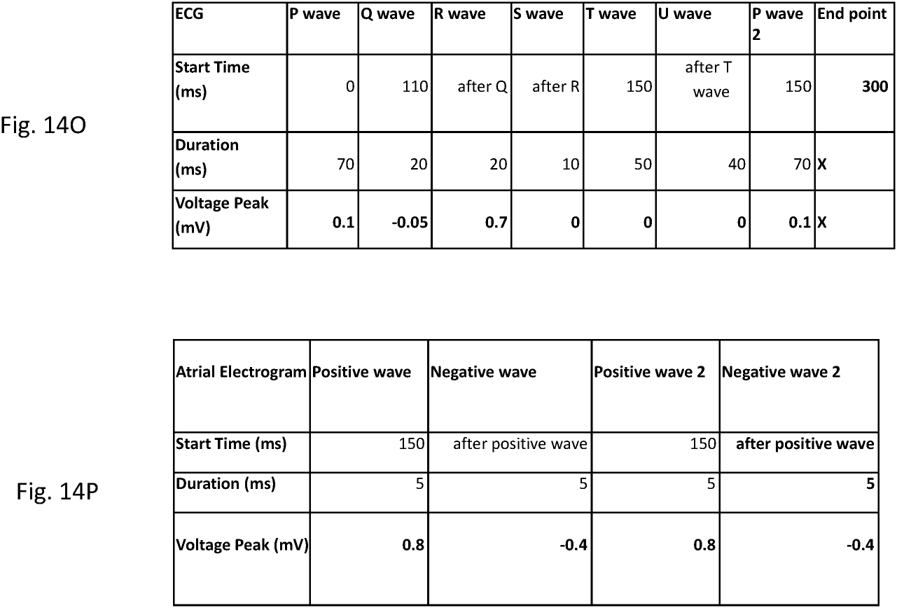

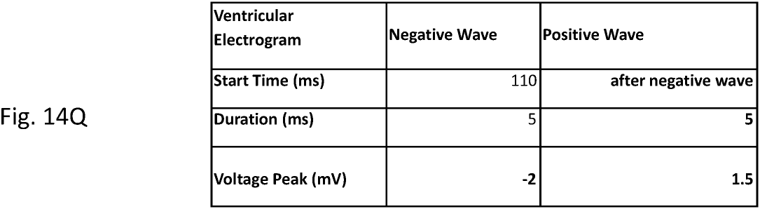

FIG. 14 depicts various signal parameters for different types of simulations and scenarios. While the signal parameters depicted in FIG. 14 are for a pediatric patient, it is to be understood that the proposed system can be used for any patients, including pediatric, adolescent, adult, elderly, etc. Parameters for a pediatric normal sinus rhythm include a heart rate of 110-150 beats per minute, a PR interval of 120 ms, and a QT interval of 490 ms. The PR interval refers to the time from onset of the P wave to the start of the QRS complex. FIG. 14A is a table that depicts ECG component amplitudes, starting times, and durations for pediatric normal sinus rhythm in accordance with an illustrative embodiment. FIG. 14B is a table that depicts atrial electrogram component amplitudes, starting times, and durations for pediatric normal sinus rhythm in accordance with an illustrative embodiment. FIG. 14C is a table that depicts ventricular electrogram component amplitudes, starting times, and durations for pediatric normal sinus rhythm in accordance with an illustrative embodiment.

Parameters for pediatric sinus bradycardia include a heart rate of less than 80 bpm, and a U wave peak of 1 mV present when heart rate is below 65. FIG. 14D is a table that depicts ECG component amplitudes, starting times, and durations for pediatric sinus bradycardia in accordance with an illustrative embodiment. FIG. 14E is a table that depicts atrial electrogram component amplitudes, starting times, and durations for pediatric sinus bradycardia in accordance with an illustrative embodiment.

Parameters for heart block were also considered. The parameters for 1^{st} degree heart block in pediatric sinus bradycardia includes a PR interval of 300 ms. FIG. 14F is a table that depicts ECG component amplitudes, starting times, and durations for pediatric 1st degree heart block in accordance with an illustrative embodiment. FIG. 14G is a table that depicts atrial electrogram component amplitudes, starting times, and durations for pediatric 1st degree heart block in accordance with an illustrative embodiment. FIG. 14H is a table that depicts ventricular electrogram component amplitudes, starting times, and durations for pediatric 1st degree heart block in accordance with an illustrative embodiment.

With respect to 2^{nd} degree heart block, Mobitz Type 1, the P-R interval increases 40 ms every beat until a QRS complex is dropped. The ratio of QRS complexes omitted can be ⅓, ¼, ⅕, etc. FIG. 14I is a table that depicts ECG component amplitudes, starting times, and durations for pediatric Mobitz type 1, 2nd degree heart block in accordance with an illustrative embodiment. The PR interval increases by 40 ms for 3 beats in a row and the fourth beat has peaks of 0 mV for all waves except the P wave. This process repeats for every four beats, creating a ratio of ⅓ (1 dropped QRS complex after every three QRS complexes). FIG. 14J is a table that depicts atrial electrogram component amplitudes, starting times, and durations for pediatric Mobitz type 1, 2nd degree heart block in accordance with an illustrative embodiment. FIG. 14K is a table that depicts ventricular EG component amplitudes, starting times, and durations for pediatric Mobitz type 1 2nd degree heart block in accordance with an illustrative embodiment. The PR interval increases by 40 ms for 3 beats in a row and the fourth beat has peaks of 0 mV for the entire ventricular EG. This process repeats for every four beats, creating a ratio of ⅓.

With respect to 2^{nd} degree heart block Mobitz Type II, the heart rate is normal, but will omit the QRS complex and T wave. The ratio of QRS intervals omitted can be ⅓, ¼, or ⅕, but the omissions may be also be random. FIG. 14L is a table that depicts ECG component amplitudes, starting times, and durations for pediatric Mobitz type 2, 2nd degree heart block in accordance with an illustrative embodiment. Every third beat has a dropped QRS complex, with peaks of 0 mV for all waves except the P wave. This process repeats for every three beats, creating a ratio of ½. FIG. 14M is a table that depicts atrial electrogram component amplitudes, starting times, and durations for pediatric Mobitz type 2, 2nd degree heart block in accordance with an illustrative embodiment. FIG. 14N is a table that depicts ventricular electrogram component amplitudes, starting times, and durations for pediatric Mobitz type 2, 2nd degree heart block in accordance with an illustrative embodiment. Every third beat has a dropped ventricular electrogram, with peaks of 0 mV. This process repeats for every three beats, creating a ratio of ½.

Parameters for pediatric atrial flutter were also considered. FIG. 14O is a table that depicts ECG component amplitudes, starting times, and durations for pediatric atrial flutter in accordance with an illustrative embodiment. Atrial flutter is a reentrant tachycardia that has a conduction ratio of 2:1 of P waves to QRS complexes, respectively. FIG. 14P is a table that depicts atrial electrogram component amplitudes, starting times, and durations for pediatric atrial flutter in accordance with an illustrative embodiment. There are two atrial impulses per cycle. FIG. 14Q is a table that depicts ventricular electrogram component amplitudes, starting times, and durations for pediatric atrial flutter in accordance with an illustrative embodiment.

Signal variations were also considered. Pediatric ECG samples of normal sinus rhythm were acquired as JPEGs from available data. These images were uploaded into the system for testing. To measure beat-to-beat heart rate variation, lines were drawn between the starts of consecutive P-waves. The lengths yielded relational measurements in the units of pixels. The mean of the lengths was found, and the maximum and minimum lengths were compared to the average to yield percent difference values. These percentages were averaged across multiple data sets from patients ranging from 2 months old to 15 years old. The ranges of lengths of an ECG cycle was determined to be the average heart rate ± 4%.

To characterize signal drift, lines were again drawn between the starts of consecutive P-waves. From each measurement, the angle between the drawn line and the horizontal was recorded. The average absolute angle was 1.6°. Because the data did not specify the axes’ scales, each x-axis division was assumed to be 40 ms and each y-axis division was assumed to be 0.1 mV, which are the standard values, respectively. Using basic geometry and an assumed heart rate of 120 beats per minute, the average absolute value of the rise of drift was determined to be 0.035 mV.

It was found that there was too much variation between ECG data to characterize ranges for specific PQRST parameters. Thus, variation parameters can be chosen by qualitative assessment in comparison with real ECG samples. For normal sinus rhythm, the ranges of each ECG component currently being used are: P-wave: +/- 0.05 mV; Q-wave: +/-0.03 mV; R-wave: +/- 0.1 mV; S-wave: +/- 0.06 mV; T-wave: +/- 0.03 mV. In alternative implementations, different values may be used. The desired intensity of random electrical noise was specified by the users, and using random Gaussian noise, the standard deviation of the desired noise level was specified to be 0.005 mV.

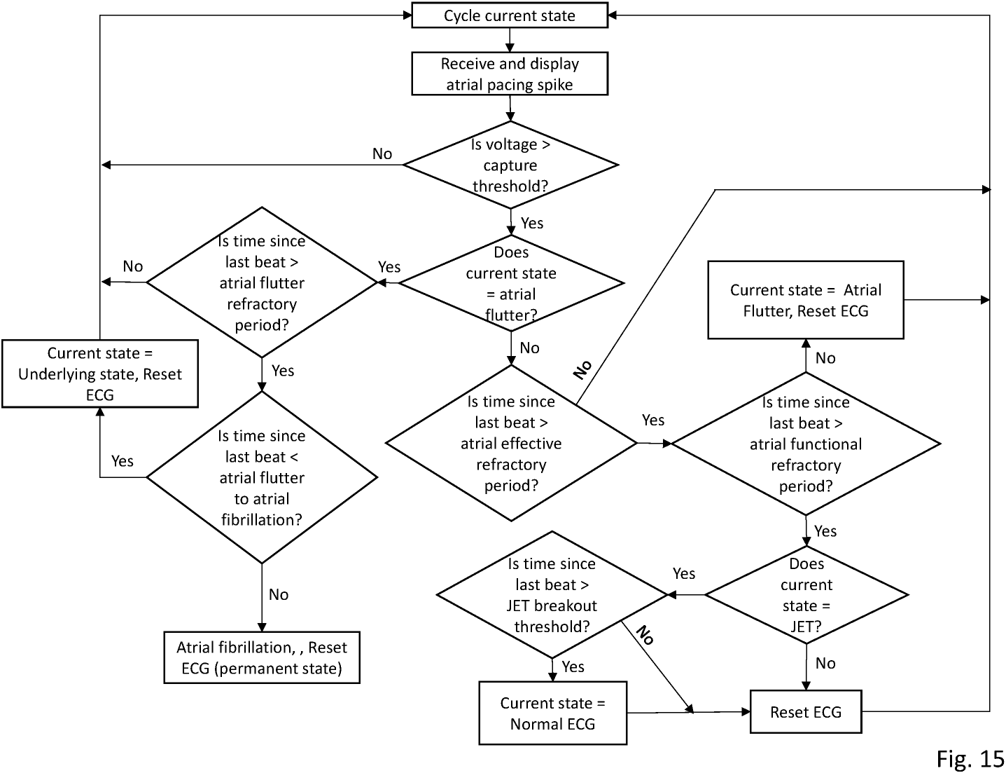

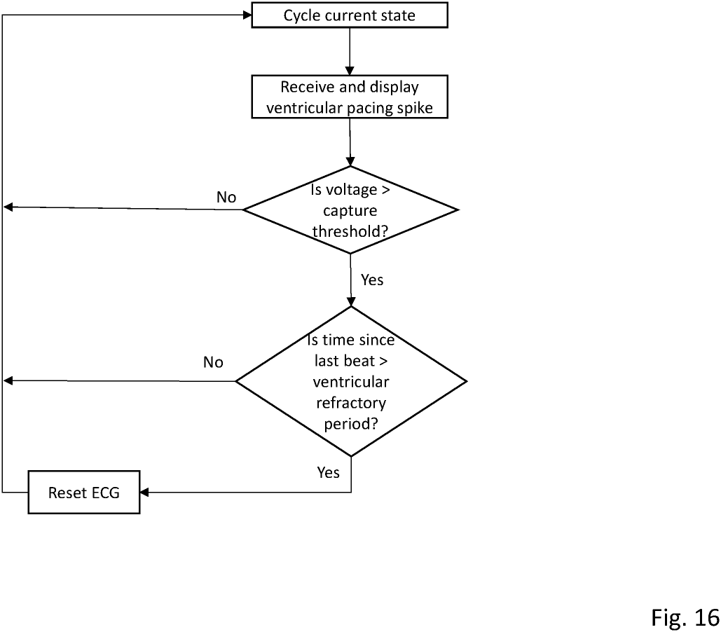

As discussed herein, decision trees can be used to determine system responses to various inputs. FIG. 15 is a flow diagram of a decision tree for atrial events in accordance with an illustrative embodiment. FIG. 16 is a flow diagram of a decision tree for ventricular events in accordance with an illustrative embodiment. In alternative embodiments, fewer, additional, and/or different operations may be performed. Also, the use of a flow diagram is not mean to be limiting with respect to the order of operations performed. As shown, the system generates ECG, atrial electrogram, and ventricular electrogram signals with real-time dynamic responses for voltages output from pacemaker depending on timing and amplitude. In FIG. 15, resetting the ECG denotes generating a P wave, and in FIG. 16 resetting the ECG denotes generating a QRS complex.

Referring to FIG. 15, the system cycles the current state, and receives and displays an atrial pacing spike. A determination of whether the signal voltage is greater than a capture threshold is made by a processor of the system. If the voltage is not greater than the capture threshold, the system continues to cycle the current state. If the voltage is greater than the capture threshold, the system determines whether the current state is atrial flutter. If the current state is determined to be atrial flutter, the system determines whether the amount of time since the last heartbeat is greater than the atrial flutter refractory period. If the amount of time since the last heartbeat is not greater than the atrial flutter refractory period, the system continues to cycle the current state. If the amount of time since the last heartbeat is greater than the atrial flutter refractory period, the system determines whether the amount of time since the last heartbeat is less than atrial flutter to atrial fibrillation. If it is determined that the amount of time since the last heartbeat is less than atrial flutter to atrial fibrillation, the system sets the current state as the underlying state, the ECG is reset, and the system continues the cycle. If it is determined that the amount of time since the last heartbeat is not less than atrial flutter to atrial fibrillation, the system sets the state to atrial fibrillation, resets the ECG, and the continues the cycle.

Referring again to the atrial flutter determination, if the current state is determined not to be atrial flutter, the system determines whether the amount of time since the last heartbeat is greater than the atrial effective refractory period. If the system determines that the amount of time since the last heartbeat is not greater than the atrial effective refractory period, the system continues to cycle the current state. If the system determines that the amount of time since the last heartbeat is greater than the atrial effective refractory period, the system determines whether the amount of time since the last heartbeat is greater than the atrial functional refractory period. If it is determined that the amount of time since the last heartbeat is not greater than the atrial functional refractory period, the system sets the current state to atrial flutter, resets the ECG, and continues the cycle. If it is determined that the amount of time since the last heartbeat is greater than the atrial functional refractory period, the system determines whether the current state is junctional ectopic tachycardia (JET). If it is determined that the current state is not JET, the system resets the ECG and the cycle continues. If it is determined that the current state is JET, the system determines whether the amount of time since the last heartbeat is greater than the JET breakout threshold. If the amount of time since the last heartbeat is not greater than the JET breakout threshold, the system resets the ECG and the cycle continues. If the amount of time since the last heartbeat is determined to be greater than the JET breakout threshold, the current state is set to normal ECG, the ECG is reset, and the cycle continues.

Referring to FIG. 16, the system cycles the current state, and receives and displays a ventricular pacing spike. A determination of whether the signal voltage is greater than a capture threshold is made by a processor of the system. If the voltage is not greater than the capture threshold, the system continues to cycle the current state. If the system determines that the voltage is greater than the capture threshold, the system determines whether the amount of time since the last heartbeat is greater than the ventricular refractory period. If it is determined that the amount of time since the last heartbeat is not greater than the ventricular refractory period, the system continues to cycle the current state. If it is determined that the amount of time since the last heartbeat is greater than the ventricular refractory period, the system resets the ECG and the cycle continues.

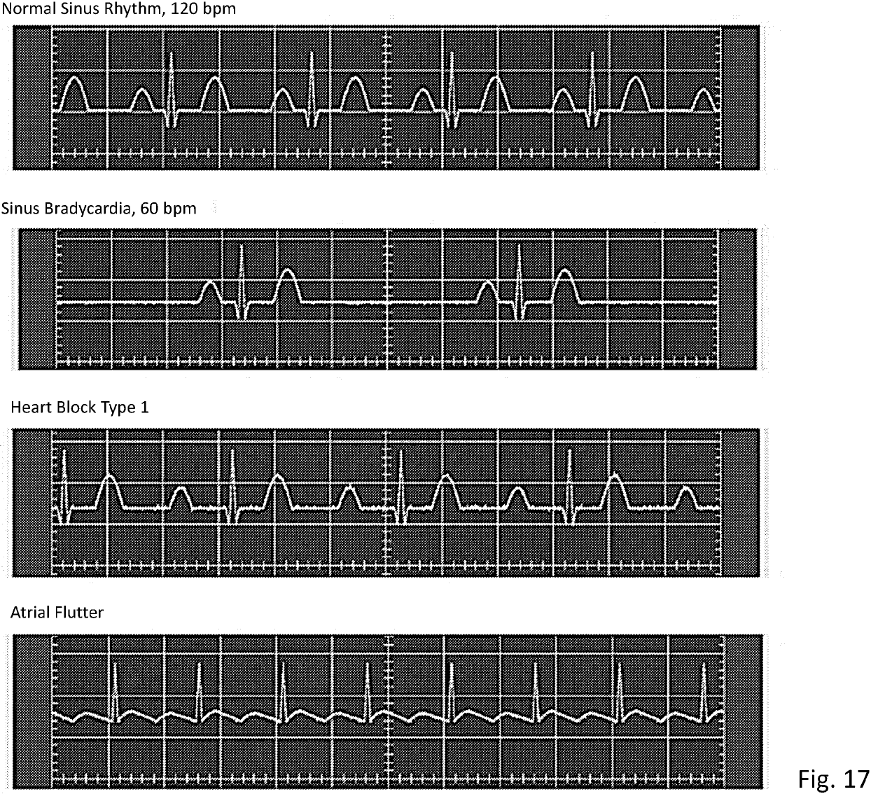

FIG. 17 depicts ECG signals for developed rhythms in accordance with an illustrative embodiment. The ECG signals shown in FIG. 17 were captured on nScope, but a different capture system may be used in alternative embodiments. More specifically, FIG. 17 shows how other rhythms, including other variations of heart block and JET, manipulate the common scaffold for rhythms presented in a normal sinus rhythm. The signals of FIG. 17 include the normal sinus rhythm (120 bpm), sinus bradycardia (60 bpm), heart block (Type 1), and atrial flutter.

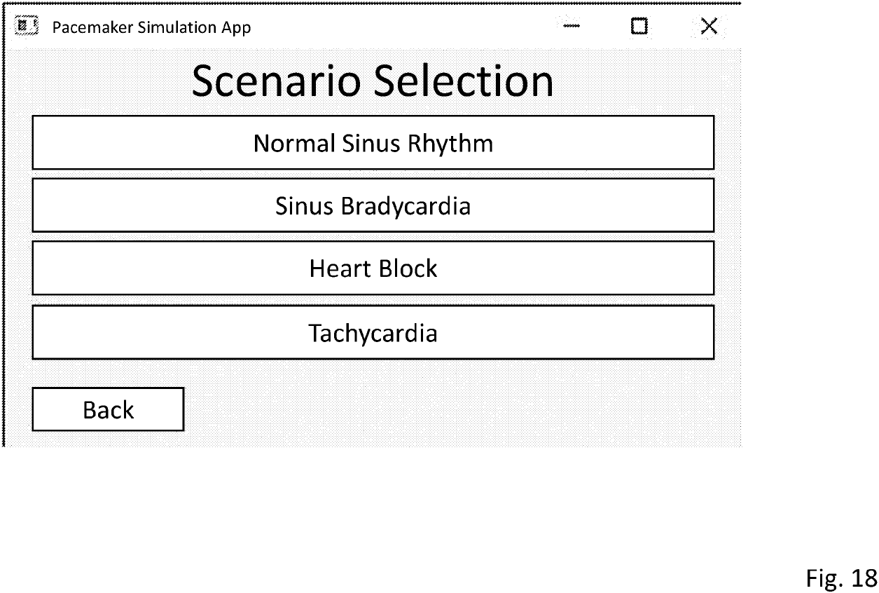

FIGS. 18-23 relate to various embodiments of the user interface and the application used to implement the user interface. In one embodiment, the UI application can have three core functions. First, it has a menu system where users navigate to different pages and enter parameter values by touching buttons and other widgets on screen. Second, it accurately sends user inputs to a controller device (e.g., a Teensy) via USB or other method. Third, it receives ECG values from the controller and displays them on a display screen smoothly without lag. Additional functions such as user performance feedback and user profile systems can also add significant value to the UI application.

In one embodiment, the UI application is implemented in Python. PyQt5, pyqtgraph and pyserial are the three Python libraries used to build the core functions. To run the application on RPI, these libraries and other dependencies were installed on the RPI first. The latest version of Raspbian OS (operating system for RPI) comes with Python3 preinstalled. Hence, the system was able to run the application directly in RPI’s Python3 shell. In alternative embodiments, different tools can be used to construct and run the user interface.

The PyQt5 library provides an extensive list of Python classes, functions, and methods for graphical user interface building. This library was chosen because of its comprehensiveness, built-in thread management, overall popularity for industrial level applications, and because of the proficiency in Python. The application was written in an object-oriented style, where similar pages in the menu system are grouped in a single Python QWidget class. When users press on interactive parts of the screen (i.e. buttons and input widgets), specific methods for that page’s QWidget class are called on the backend to process user actions and perform necessary functions.

User inputs to the system can include the scenario selected, all ECG parameters, and all EG parameters. In the UI application, these user inputs are stored as global variables. Before the user inputs are sent to the controller (e.g., Teensy), the parameters are assembled into a standardized string format. In ‘Demo’ mode, this parameters string is sent to the controller when the user presses the ‘Enter’ button on a ‘Parameters’ page. In ‘Practice’ mode, the randomly generated parameters are sent when the ‘Practice’ mode button is pressed.

In some embodiments, the controller (e.g., Teensy) used does not have a set baud rate. Instead, it always tries to run at full USB speed, which is 480 Mbps for a USB 2.0 cable. Information between these devices are always sent as byte strings. The main loop in the controller is set up such that it only runs the ECG simulation code when there is nothing to read in the USB buffer. When the RPI sends the parameters string to the controller, it is immediately parsed by the controller, which stores the parameters as global variables. These global variables are then used when the ECG simulation begins.

In order to get a smooth ECG display on the ‘Graphing’ page, the RPI can read ECG values from the controller and update the ECG plot with as little latency between the two as possible. This presents a common challenge for UI design, where there are background functions that are constantly running that will interfere with the main UI code that displays the graphical interface. The main UI code can be constructed as a blocking function that prevents the execution of other parts of the code unless it is told to do so. For small background functions that can be executed quickly, one can switch between running the main UI code and the small background function without the user noticing. However, for long-running functions such as the one needed to constantly read the controller and update the ECG plot, executing this function will cause the main UI code to become stuck and unresponsive. To prevent this, threading is used. Threading separates the executions of the main UI code and controller communication code into separate threads for the main CPU to run. This allows both functions to run essentially simultaneously, so that when a new value is read from the controller, the ECG plot is updated and the change is displayed on the screen. PyQt5 provides the QThread and QRunnable classes to easily implement threading. The pyqtgraph library was used to make a graph widget that can be embedded into ‘Graphing’ QWidget class to display the ECG plot. In alternative embodiments, different software and/or hardware may may be used.

The controller calculates ECG values and sends them to the main processor via USB. In the code there is a 5 ms delay between sending each value. The 5 ms delay was determined through trial and error by seeing if the ECG display was smooth on screen. If the delay was reduced below 5 ms, the main processor would receive two or more ECG values at once and encounter an error when parsing the values. This is because the main processor takes about 5 ms to execute the code between each read, and if the buffer is not cleared by the time the controller sends the next value, the buffer will be filled by the previous and new values.

FIGS. 18-23 are various screen shots that show the workflow for various modes of the system. For the Practice mode, a Graphing page is displayed immediately when this mode is entered, and a Post Practice Selection page can be shown when the user quits the simulation. FIG. 18 is a scenario selection page of the UI application in accordance with an illustrative embodiment. With the scenario selection page, the user can choose from one of a plurality of different scenarios. For example, pressing the “Heart Block” button leads to another page with four heart block scenarios to select from and pressing the “Tachycardia” button leads to a different page with three tachycardia scenarios to select from. Pressing the “Back” button returns to the Main Menu page.

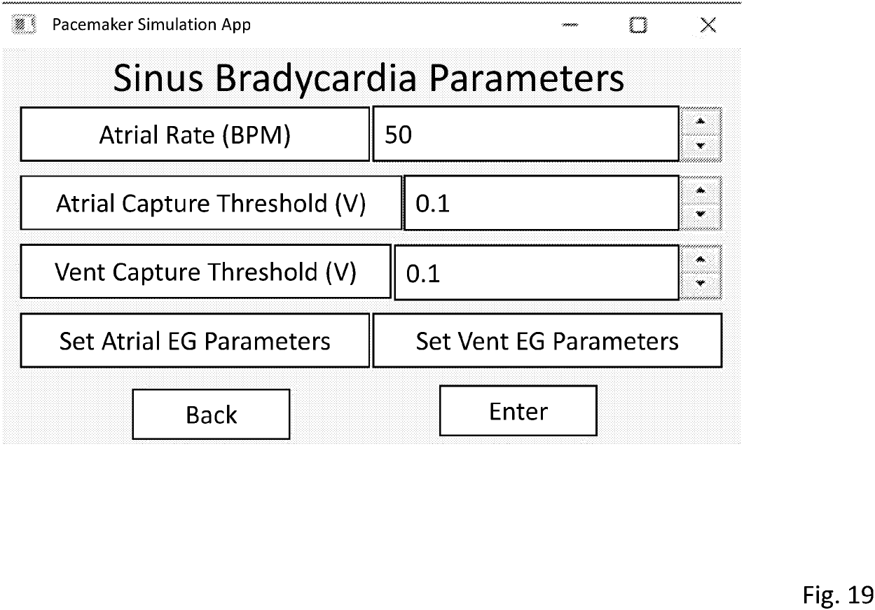

FIG. 19 is a sinus bradycardia parameters page of the user interface in accordance with an illustrative embodiment. The parameters page for each scenario is slightly different as they will have different attributes that can be changed. All parameters pages can have an ‘Atrial Capture Threshold’ value, a ‘Ventricular Capture Threshold’ value, a ‘Set Atrial EG Parameters’ option, and a ‘Set Vent EG Parameters’ option as these are common among all scenarios. Users can change parameter values by pressing the up or down arrow buttons. The increment value of pushing on up or down buttons once is set to the resolution of that parameter. Up and down buttons no longer change values when the upper and lower bounds of that parameter are reached. Users can press and hold on up and down buttons to change values more quickly. Pressing the “Back” button returns to the Scenario Selection page. Pressing the “Enter” button leads to a Graphing page.

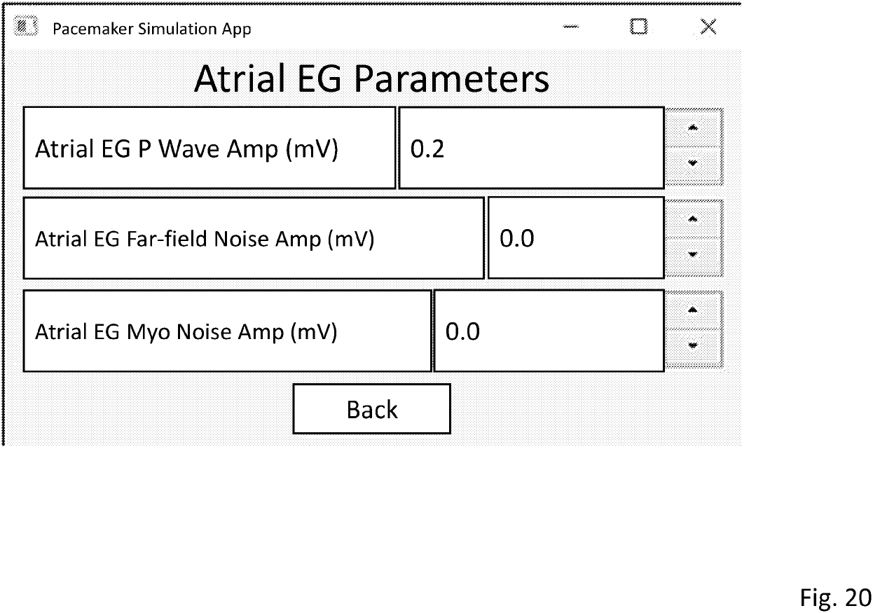

FIG. 20 is an atrial EG parameters page of the user interface in accordance with an illustrative embodiment. This page is displayed when the “Set Atrial EG Parameters” button is pressed on any of the parameters pages. This page is the same regardless of scenario selected. Pressing the “Back” button returns to the parameters page from which it was called.

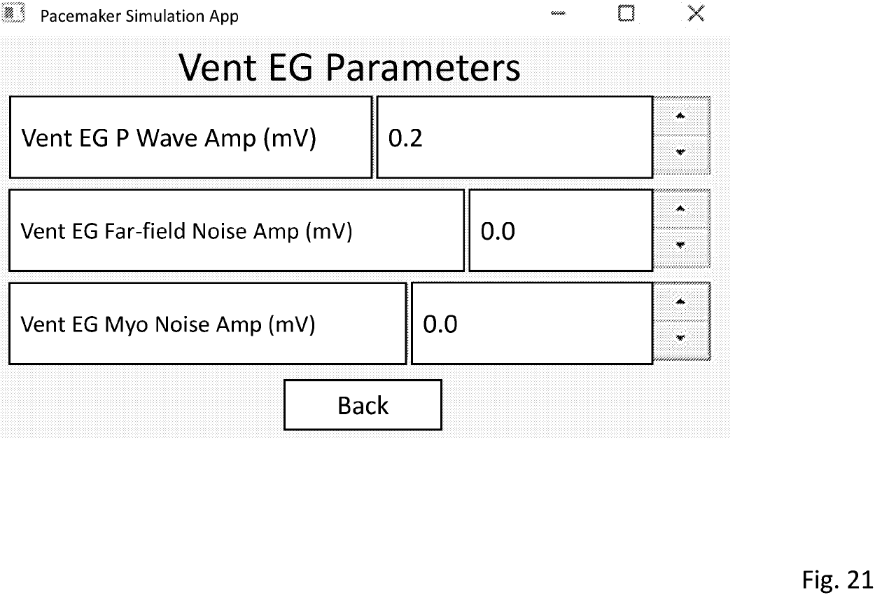

FIG. 21 is a ventricular EG parameters page of the user interface in accordance with an illustrative embodiment. This page is displayed when the “Set Vent EG Parameters” button is pressed on any of the parameters pages. This page is the same regardless of scenario selected. Pressing the “Back” button returns to the parameters page from which the ventricular EG parameters page was called.

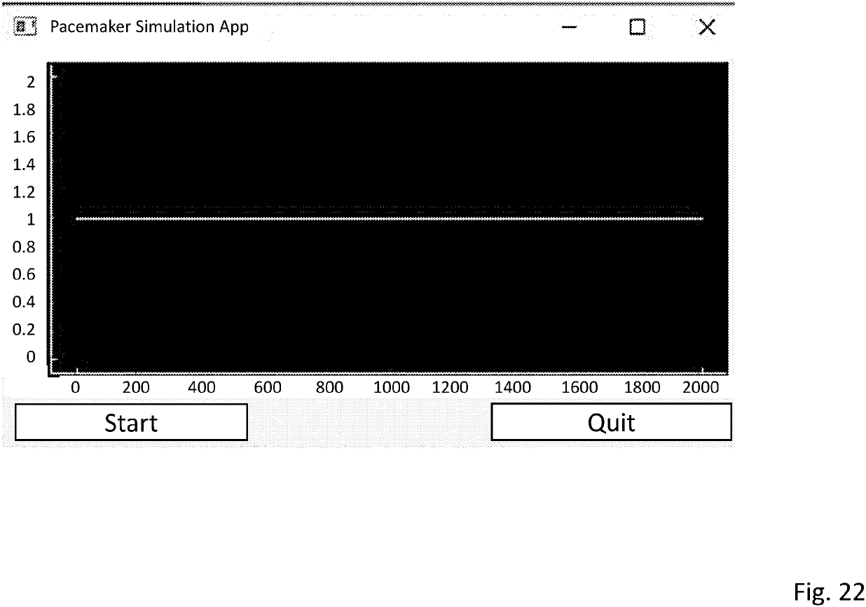

FIG. 22 depicts a graphing page in accordance with an illustrative embodiment. Pressing the “Start” button begins the simulation and the ECG for the user-specified scenario begins displaying. When the user begins pacing, the ECG will change accordingly. Pressing the “Quit” buttons stops the simulation.

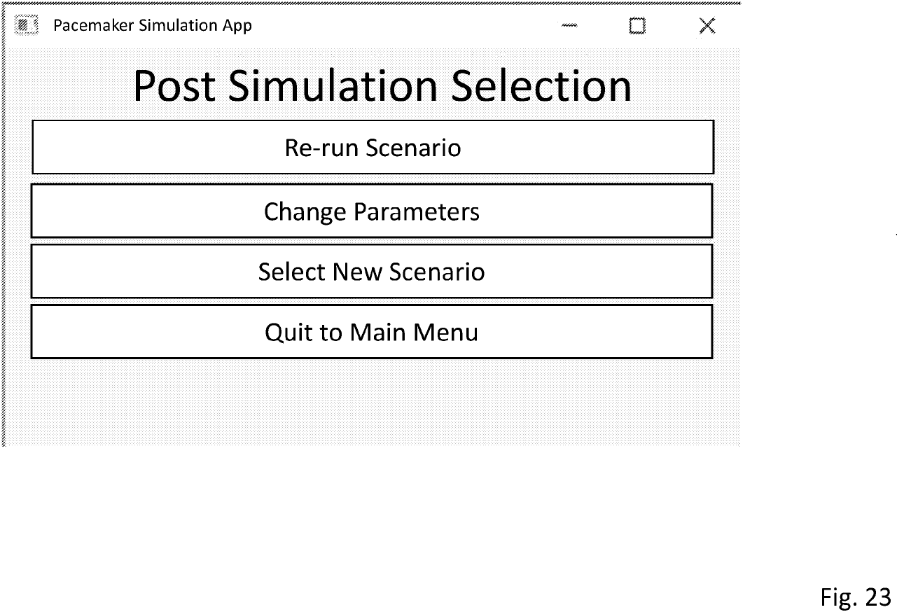

FIG. 23 is a post simulation selection page of the user interface in accordance with an illustrative embodiment. In one embodiment, after quitting a simulation, the user has these four options to choose from. Starting from top to bottom, the first button brings back the previous Graphing page with the same scenario, the second button returns to the parameters page for the same scenario, the third button returns to the Scenario Selection page, and the last button returns to the Main Menu page.

The embodiments depicted in FIGS. 18-23 are meant to be examples, and are not meant to be limiting with respect to the implementation of the user interface. In alternative embodiments, different user interface screens, options, and layouts may be used. Similarly, the specific hardware referenced in the specification is meant only as examples, and different hardware and configurations may be used in alternative embodiments.

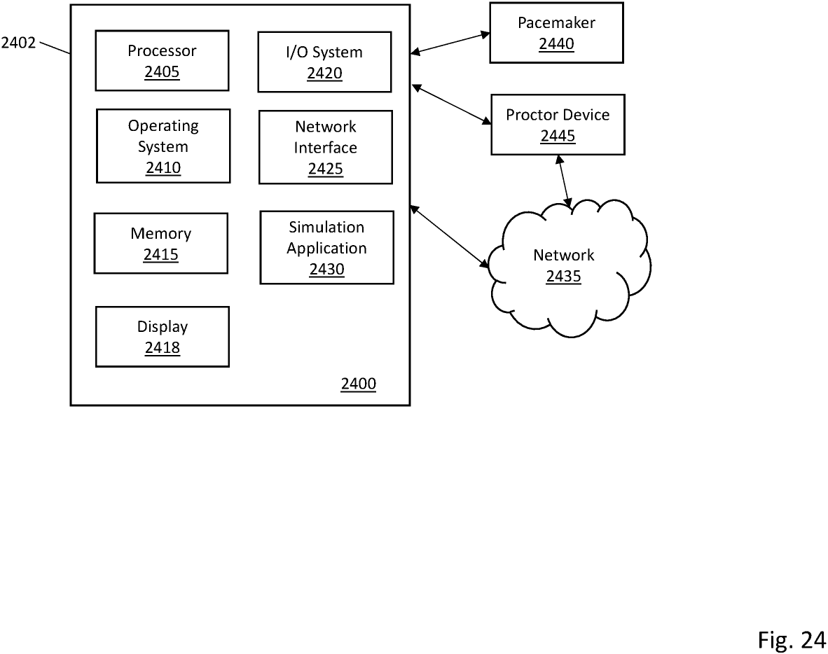

FIG. 24 is a block diagram of a computing system 2400 to implement the cardiac conduction simulation system for pacemakers in accordance with an illustrative embodiment. The computing system 2400 includes a housing 2402. The housing 2402 can be made from acrylonitrile butadiene styrene (ABS), rubber, etc. The housing may include padding for additional support and protection of the system, and can also include handles for easy movement. The housing 2402 can provide access to system ports such as one or more communication/data ports, one or more networking ports, one or more pacemaker connection ports, one or more power charging or delivery ports, etc.

FIG. 24 depicts the computing device 2400 in communication with a network 2435, a pacemaker 2440, and a proctor device 2445. The pacemaker 2440 can be any type of pacemaker. The proctor device 2445 can be any type of computing device operated by a proctor/trainer for the system, and can include a processor, memory, transceiver, user interface, etc. The proctor device 2445 can be used by the proctor to monitor and control the simulations as described herein. The proctor device 2445 can communicate directly with the computing device 2400, or indirectly through the network 2435, depending on the implementation.

The computing device 2400 (i.e., simulation system) includes a processor 2405, an operating system 2410, a memory 2415, a display 2418, an input/output (I/O) system 2420, a network interface 2425, and a simulation application 2430. In alternative embodiments, the computing device 2400 may include fewer, additional, and/or different components. The components of the computing device 2400 communicate with one another via one or more buses or any other interconnect system. The computing device 2400 can be any type of computing device (e.g., smartphone, tablet, laptop, desktop, etc.), including a dedicated standalone computing system that is designed to perform the simulations.

The processor 2405 can be in electrical communication with and used to control any of the system components described herein. For example, the processor can be used to execute the simulation application 2430, process received user selections, send commands to the pacemaker 2440, receive signals from the pacemaker, process the data using decision trees or other methods, display results, etc. The processor 2405 can be any type of computer processor known in the art, and can include a plurality of processors and/or a plurality of processing cores. The processor 2405 can include a controller, a microcontroller, an audio processor, a graphics processing unit, a hardware accelerator, a digital signal processor, etc. Additionally, the processor 2405 may be implemented as a complex instruction set computer processor, a reduced instruction set computer processor, an x86 instruction set computer processor, etc. The processor 2405 is used to run the operating system 2410, which can be any type of operating system.

The operating system 2410 is stored in the memory 2415, which is also used to store programs, user data, pacemaker readings, network and communications data, peripheral component data, the simulation application 2430, and other operating instructions. The memory 2415 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 2415 can be in the cloud to provide cloud storage for the system. Similarly, in one embodiment, any of the computing components described herein (e.g., the processor 2405, etc.) can be implemented in the cloud such that the system can be run and controlled through cloud computing.

The I/O system 2420 is the framework which enables users and peripheral devices to interact with the computing device 2400. The display 2418 can include a touch screen in some embodiments, and the touch screen can be part of the I/O system 2420 that allows the user/proctor to make selections. The display 2418 can be any type of display, and can be used to present user interface screens and pacemaker readings to the user. The I/O system 2420 can also include one or more speakers, one or more microphones, a keyboard, a mouse, one or more buttons or other controls, etc. that allow the user to interact with and control the computing device 2400. The I/O system 2420 also includes circuitry and a bus structure to interface with peripheral computing devices such as power sources, universal service bus (USB) devices, data acquisition cards, peripheral component interconnect express (PCIe) devices, serial advanced technology attachment (SATA) devices, high definition multimedia interface (HDMI) devices, proprietary connection devices, etc.

The network interface 2425 includes transceiver circuitry (e.g., a transmitter and a receiver) that allows the computing device 2400 to transmit and receive data to/from other devices such as the proctor device 2445, other remote computing systems, servers, websites, etc. The network interface 2425 enables communication through the network 2435, which can be one or more communication networks. The network 2435 can include a cable network, a fiber network, a cellular network, a wi-fi network, a landline telephone network, a microwave network, a satellite network, etc. The network interface 2425 also includes circuitry to allow device-to-device communication such as Bluetooth® communication.

The simulation application 2430 can include software and algorithms in the form of computer-readable instructions which, upon execution by the processor 2405, performs any of the various operations described herein such as displaying screens of the user interface on the display 2418, receiving user data, processing user selections, sending commands to the pacemaker, processing pacemaker responses, presenting an EKG and other results, processing commands from the proctor device 2445, etc. The simulation application 2430 can utilize the processor 2405 and/or the memory 2415 as discussed above. In an alternative implementation, the simulation application 2430 can be remote or independent from the computing device 2400, but in communication therewith.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A cardiac conduction simulation system for pacemakers comprising:
a memory configured to store one or more inputs from one or more users, wherein the one or more inputs include a selection of an arrhythmia scenario for a pacemaker; and
a processor operatively coupled to the memory and configured to:
generate an electrogram signal for the pacemaker based at least in part on the selected scenario;
process one or more output pacing signals received from the pacemaker; and
generate an electrocardiogram (ECG) signal in accordance with the processed one or more output pacing signals from the pacemaker.

2. The system of claim 1, wherein the scenario comprises a heart condition that is affected by the pacemaker.

3. The system of claim 1, wherein the one or more inputs include a parameter setting for the scenario, and wherein the parameter setting includes an atrial rate and a ventricular rate.

4. The system of claim 3, wherein the parameter setting includes atrial or ventricular association or dissociation, and wherein the parameter setting includes a PR interval in a case of ventricular association.

5. The system of claim 3, wherein the parameter setting includes one or more capture thresholds.

6. The system of claim 1, further comprising an analog to digital converter (ADC) in communication with the processor, wherein the ADC is configured to measure of a voltage of the one or more output pacing signals of the pacemaker.

7. The system of claim 1, further comprising a digital to analog converter (DAC) controlled by the processor, wherein the DAC is configured to generate the electrogram signal.

8. The system of claim 1, wherein the processor is configured to temporally align the electrogram signal and the ECG signal.

9. The system of claim 1, further comprising an initial heart state stored in the memory, wherein the processor transitions a simulation from the initial heart state to a subsequent heart state based at least in part on the one or more output pacing signals of the pacemaker that are received.

10. The system of claim 9, wherein the ECG signal corresponds to the subsequent heart state.

11. The system of claim 1, wherein the processor is configured to add electrical noise to the electrogram signal that is received by the pacemaker.

12. The system of claim 1, wherein the processor is configured to add one or more of digital noise, drift, and randomization to the ECG signal.

13. The system of claim 1, further comprising a display operatively coupled to the processor, wherein the display is configured to:
display the ECG signal;
display of an ECG effective heart rate; and
display performance feedback data to the one or more users during or after completion of a simulation.

14. The system of claim 1, further comprising a pacemaker circuit that is configured to interface with the pacemaker, wherein the pacemaker circuit includes a load resistor configured to simulate resistance in a human heart.

15. The system of claim 14, further comprising a sensing circuit that receives an output from the pacemaker circuit, wherein the sensing circuit includes an inverted operational amplifier to divide and invert the output from the pacemaker circuit such that a maximum voltage of the output is controlled.

16. The system of claim 15, wherein the sensing circuit further comprises a noninverting operational amplifier that is configured to rectify and amplify the output such that the output is maintained at a specific voltage.

17. A method of simulating cardiac conduction for pacemaker management, the method comprising:
storing, in a memory of a computing system, one or more inputs from one or more users, wherein the one or more inputs include a selection of an arrhythmia scenario for a pacemaker;
generating, by a processor operatively coupled to the memory, an electrogram signal for the pacemaker based at least in part on the selected scenario;
processing, by the processor, one or more output pacing signals received from the pacemaker; and
generating, by the processor, an electrocardiogram (ECG) signal in accordance with the processed one or more output pacing signals from the pacemaker.

18. The method of claim 17, further comprising storing, in the memory, a parameter setting for the scenario, wherein the parameter setting includes an atrial rate, a ventricular rate, atrial association or dissociation, ventricular association or dissociation, a P wave amplitude, an R wave amplitude, or one or more capture thresholds.

19. The method of claim 17, further comprising temporally aligning, by the processor, the electrogram signal, and the ECG signal.

20. The method of claim 17, further comprising adding, by the processor, electrical noise to the electrogram signal.
